(19) United States
(12) Patent Application Publication
(10) Pub. No.: US 2024/0277624 A1
(43) Pub. Date: Aug. 22, 2024

(54) NEW FORMULATIONS COMPRISING BIOLOGIC ACTIVE DRUGS

(71) Applicant: NANEXA AB, Uppsala (SE)

(72) Inventors: Anders JOHANSSON, Uppsala (SE); Mårten ROOTH, Uppsala (SE); Erik LINDAHL, Uppsala (SE); Joel HELLRUP, Uppsala (SE); Jonas FRANSSON, Uppsala (SE); David WESTBERG, Uppsala (SE); Otto SKOLLING, Täby (SE)

(21) Appl. No.: 18/568,363

(22) PCT Filed: Jun. 10, 2022
(86) PCT No.: PCT/GB2022/051461

§ 371 Date: Dec. 8, 2023

(30) Foreign Application Priority Data
Jun. 10, 2021 (GB) 2108282.1

(51) Int. Cl. A61K 9/50; A61K 9/00; A61K 9/10; A61K 9/51; A61K 38/00; C23C 16/40; C23C 16/44; C23C 16/455

(52) U.S. Cl. CPC A61K 9/5073; A61K 9/0019; A61K 9/10; A61K 9/501; A61K 9/5115; C23C 16/407; C23C 16/4417; C23C 16/45555; A61K 38/00

(57) ABSTRACT

There is provided a pharmaceutical formulation that is useful in delivering a biologic active drug in a high dose, comprising a plurality of particles suspended in an aqueous carrier system, which particles: pharmaceutical formulation, comprising a plurality of particles suspended in a carrier system, which particles: (a) have a weight-, number-, or volume-based mean diameter that is between amount 10 nm and about 700 pm; and (b) comprise solid cores comprising a biologic active drug, coated, at least in part, by a coating of inorganic material, and wherein the formulation comprises a concentration of said biologic active drug of at least 10 mg/ml. Preferred biologic active drug include immunoglobulins, monoclonal antibodies, antibody mimetics, cytokines and cytokine antagonists, and human peptide hormones.

### FIELD OF THE INVENTION

This Invention relates to a new formulation for use in for example the field of drug delivery of biologic active drugs, including immunoglobulins, antibodies, antibody mimetics, cytokines and cytokine antagonists, and human peptide hormones.

### PRIOR ART AND BACKGROUND

The listing or discussion of an apparently prior-published document in this specification should not necessarily be taken as an acknowledgement that the document is part of the state of the art or common general knowledge.

In the field of drug delivery, biologic active drugs/active pharmaceutical ingredients (APIs), such as immunoglobulins, antibodies, and antibody analogues and mimetics have increasingly proven to be useful in the treatment of a broad range of diseases and disorders.

Compared to other APIs, antibodies are not very potent and may require injection of doses of typically 50 to 500 mg per injection daily, weekly, every other week, or with even larger intervals.

One of the major problems with using biologic active drugs is the inability to form an injectable formulation with high drug load with regard to the API. Many of those APIs form a gel when the concentration increases.

In the case of any sustained release composition, it is of critical importance that its release profile shows minimal initial rapid release of active ingredient, that is a large concentration of drug in plasma shortly after administration. Such a ‘burst’ release will result in unwanted, high concentrations active ingredient, and may be hazardous in the case of drugs that have a narrow therapeutic window. It is also important to ensure that active ingredients are released at a desired and predictable rate in vivo following administration, in order to ensure the optimal pharmacokinetic profile.

In the case of injectable suspensions of biologic active pharmaceutical ingredients (API), such as protein formulations, including e.g. antibody formulations, it is often desirable to obtain high concentration of said proteins e.g. due to the relatively low potency of antibodies and other proteins and the associated need to be able to provide high doses of said protein formulations.

High concentration protein formulation (HCPF) is a term that refers to such formulations, and though no clear definition of the concentration has been agreed on, typical ranges are between 50 and 150 mg/mL for monoclonal antibody (mAb) drugs. HCPFs carry with them undesirable properties differing from those of formulations of low protein concentration, such as a tendency of lower stability, tendency of gel formation of the compounds, and an increased propensity for protein particle formation,

There is thus a general need in the art for effective and/or improved drug transport and delivery systems for formulations comprising biologic active drugs such as antibody drugs in high concentrations.

Atomic layer deposition (ALD) is a technique that is employed to deposit thin films comprising a variety of materials, including organic, biological, polymeric and, especially, inorganic materials, such as metal oxides, on solid substrates. It is an enabling technique for atomic and close-to-atomic scale manufacturing (ACSM) of materials, structures, devices and systems in versatile applications (see, for example, Zhang et al. Nanomanuf. Metrol. 2022, https://doi.org/10.1007/s41871-022-00136-8). Based on its self-limiting characteristics, ALD can achieve atomic-level thickness that is only controlled by adjusting the number of growth cycles. Moreover, multilayers can be deposited, and the properties of each layer can be customized at the atomic level.

Due to its atomic-level control, ALD is used as a key technique for the manufacturing of, for example, next-generation semiconductors, or in atomic-level synthesis of advanced catalysts as well as in the precise fabrication of nanostructures, nanoclusters, and single atoms (see, for example, Zhang et al. vide supra).

The technique is usually performed at low pressures and elevated temperatures. Film coatings are produced by alternating exposure of solid substrates within an ALD reactor chamber to vaporized reactants in the gas phase. Substrates can be silicon wafers, granular materials or small particles (e.g. microparticles or nanoparticles).

The coated substrate is protected from chemical reactions (decomposition) and physical changes by the solid coating. ALD can also potentially be used to control the rate of release of the substrate material within a solvent, which makes it of potential use in the formulation of active pharmaceutical ingredients.

In ALD, a first precursor, which can be metal-containing, is fed into an ALD reactor chamber (in a so called ‘precursor pulse’), and forms an adsorbed atomic or molecular monolayer at the surface of the substrate. Excess first precursor is then purged from the reactor, and then a second precursor, such as water, is pulsed into the reactor. This reacts with the first precursor, resulting in the formation of a monolayer of e.g. metal oxide on the substrate surface. A subsequent purging pulse is followed by a further pulse of the first precursor, and thus the start of a new cycle of the same events (a so called ‘ALD cycle’).

The thickness of the film coating is controlled by inter alia the number of ALD cycles that are conducted.

In a normal ALD process, because only atomic or molecular monolayers are produced during any one cycle, no discernible physical interface is formed between these monolayers, which essentially become a continuum at the surface of the substrate.

In international patent application WO 2014/187995, a process is described in which a number of ALD cycles are performed, which is followed by periodically removing the resultant coated substrates from the reactor and conducting a re-dispersion/agitation step to present new surfaces available for precursor adsorption.

The agitation step is done primarily to solve a problem observed for nano- and microparticles, namely that, during the ALD coating process, aggregation of particles takes place, resulting in ‘pinholes’ being formed by contact points between such particles. The re-dispersion/agitation step was performed by placing the coated substrates in water and sonicating, which resulted in deagglomeration, and the breaking up of contact points between individual particles of coated active substance.

The particles were then loaded back into the reactor and the steps of ALD coating of the powder, and deagglomerating the powder were repeated 3 times, to a total of 4 series of cycles. This process has been found to allow for the formation of coated particles that are, to a large extent, free of pinholes (see also, Hellrup et al., Int. J. Pharm., 529, 116 (2017)).

In the case of an injectable suspension of an active ingredient, it is also important that the size of the suspended particles is controlled so that they can be injected through a needle. If large, aggregated particles are present, they will not only block the needle, through which the suspension is to be injected, but also will not form a stable suspension within (i.e. they will instead tend to sink to the bottom of) the injection liquid.

We provide novel, injectable biologic active drug formulations, in which ALD is used to coat drug microparticles with an inorganic coating layer, which coated particles are suspended in a vehicle, wherein the drug is one or more biologic active drugs. These formulations produce an advantageous pharmacokinetic profile by releasing active ingredient over an extended period of time to provide a therapeutically-effective level of drug in systemic circulation, without any significant initial burst effect.

We also provide pharmaceutical formulations obtained by the methods disclosed herein that enable high concentrations of APIs such as in HCPFs while reducing the negative properties associated with formulations with APIs in high concentrations.

### DISCLOSURE OF THE INVENTION

According to a first aspect of the invention there is provided a pharmaceutical formulation that is useful in delivering a biologic active drug (API) in a high dose, comprising a plurality of particles suspended in a carrier system, which particles:
(a) have a weight-, number-, or volume-based mean diameter that is between amount 10 nm and about 700 μm; and
(b) comprise solid cores comprising a biologic active drug, coated, at least in part, by a coating of inorganic material, which formulations comprise a concentration of said API of at least about 10 mg/ml and are hereinafter referred to as ‘the formulations of the invention’.

The term ‘solid’ will be well understood by those skilled in the art to include any form of matter that retains its shape and density when not confined, and/or in which molecules are generally compressed as tightly as the repulsive forces among them will allow. The solid cores have at least a solid exterior surface onto which a layer of coating material can be deposited. The interior of the solid cores may be also solid or may instead be hollow. For example, if the particles are spray dried before they are placed into the reactor vessel, they may be hollow due to the spray drying technique.

Formulations of the invention are preferably pharmaceutical formulations, in which case the formulations may comprise a pharmacologically-effective amount of a biologic active drug. Furthermore, said solid cores preferably comprise said biologic active drug.

In this respect, the solid cores may consist essentially of, or comprise, biologic active drug or agent (which drug or agent may hereinafter be referred to interchangeably as an ‘active pharmaceutical ingredient (API)’ and/or an ‘active ingredient’). Biologic active drugs also include biopharmaceuticals and/or biologics. Biologic active drugs can also include a mixture of different APIs, as different API particles or particles comprising more than one API. It is contemplated that the biologic active drug according to the invention is selected from the group comprising immunoglobulins, antibodies, monoclonal antibodies, and antibody mimetics.

The drug load comprised in a formulation of this invention may be determined as the dry drug load, which refers to the drug load as the weight percentage of the particles comprised in the formulations, and as the suspension drug load which refers to the solid contents as a weight percentage of the formulations of the invention in the form of suspensions.

Formulations of the invention comprise a high dry drug load such as about 20% or more by weight, such as about 30% or more, such as about 40% or more, such as about 50% or more, such as about 60% or more, such as about 70% or more, such as about 80% or more, such as about 90% or more, such as about 95% or more, such as at least about 99%.

Formulations of the invention comprise suspensions with a solid content of about 20% or more, such as about 30% or more, such as about 40% or more, such as about 50% or more, such as about 60% or more, such as about 70% or more, such as about 80%. The solid contents then include the APIs as well as the coating used for the particles of the formulations, why the total drug load in a suspension is affected by the dry drug load of the particles. Thus, the total drug load may be lower than the solid content of a suspension.

As described in more detail hereinafter, formulations of the invention may be in the form of a liquid, a sol, a paste, or a gel, administrable via a surgical administration apparatus that forms a depot formulation. It is contemplated that increasing the solid contents of the formulations of the invention will increase the likelihood of the formulation being in the form of an injectable paste.

Formulations of the invention may comprise a concentration of an API of at least about 10 mg/mL, such as at least about 25 mg/mL, such as at least about 50 mg/mL, such as at least about 100 mg/mL, such as at least about 150 mg/mL, such as at least about 200 mg/mL, such as at least about 250 mg/mL, such as at least about 300 mg/mL, such as at least about 350 mg/mL, such as at least about 400 mg/mL, such as at least about 450 mg/mL, such as at least about 500 mg/mL

Desirable APIs and formulations of the invention may comprise immunoglobulins, such as those that exist with ATC (Anatomical Therapeutic Chemical) codes within J06BA and J06BB, such as IgG for use in substitution and/or replacement therapies in immunodeficiency diseases and immune modulation. Such substitution or replacement therapies are used in the treatment of various neuropathies, Guillan-Barré syndrome, Kawasaki disease, and in patients suffering from cancers including lymphomas, such as B-cell non-Hodgkin lymphoma, Hodgkin lymphoma, leukaemia such as chronic lymphocytic leukaemia (CLL), and/or patients treated for a cancer by undergoing chemotherapy treatment that reduces the number of B cells or destroys B cells. Examples of such treatment include treatment with e.g. rituximab.

Immunoglobulin substitution/replacement therapy is also used in immune thrombocytopenia and autoimmune haemolytic anaemia, especially in patients that have not responded positively to other treatments.

Immunoglobulin substitution/replacement therapy is also used in patients suffering from a primary immunodeficiency, such as common variable immune deficiency (CVID) and X-linked agammaglobulinemia.

Non-limiting examples of immunoglobulins which may be used according to the present invention are immunoglobulins, normal human, for extravascular administration (J06BA01), immunoglobulins, normal human, for intravascular administration (J06BA02), anti-D (rh) immunoglobulin (J06BB01), tetanus immunoglobulin (J06BB02), varicella/zoster immunoglobulin (J06BB03), hepatitis B immunoglobulin (J06BB04), rabies immunoglobulin (J06BB05), rubella immunoglobulin (J06BB06), vaccinia immunoglobulin (J06BB07), staphylococcus immunoglobulin (J06BB08), cytomegalovirus immunoglobulin (J06BB09), diphtheria immunoglobulin (J06BB10), hepatitis A immunoglobulin (J06BB11), encephalitis, tick borne immunoglobulin (J06BB12), pertussis immunoglobulin (J06BB13), morbilli immunoglobulin (J06BB14), parotitis immunoglobulin (J06BB15), palivizumab (J06BB16), motavizumab (J06BB17), raxibacumab (J06BB18), bezlotoxumab (J06BB21), obiltoxaximab (J06BB22), anthrax immunoglobulin (J06BB19), combinations (J06BB30), or a mixture of any of these.

Desirable APIs and formulations of the invention may comprise monoclonal antibodies. Non-limiting examples of (monoclonal) antibodies which may be used according to the present invention as edrecolomab (L01XC01), rituximab (L01XC02), trastuzumab (L01XC03), gemtuzumab ozogamicin (L01XC05), cetuximab (L01XC06), bevacizumab (L01XC07), panitumumab (L01XC08), catumaxomab (L01XC09), ofatumumab (L01XC10), ipilimumab (L01XC11), brentuximab vedotin (L01XC12), pertuzumab (L01XC13), trastuzumab emtansine (L01XC14), obinutuzumab (L01XC15), dinutuximab beta (L01XC16), nivolumab (L01XC17), pembrolizumab (L01XC18), blinatumomab (L01XC19), ramucirumab (L01XC21), necitumumab (L01XC22), elotuzumab (L01XC23), daratumumab (L01XC24), mogamulizumab (L01XC25), inotuzumab ozogamicin (L01XC26), olaratumab (L01XC27), durvalumab (L01XC28), bermekimab (L01XC29), avelumab (L01XC31), atezolizumab (L01XC32), cemiplimab (L01XC33), moxetumomab pasudotox (L01XC34), tafasitamab (L01XC35), enfortumab vedotin (L01XC36), polatuzumab vedotin (L01XC37), isatuximab (L01XC38), belantamab mafodotin (L01XC39), dostarlimab (L01XC40), trastuzumab deruxtecan (L01XC41), phosphorylcholine monoclonal antibody, Bi-specific T-cell Engagers (BITE); such as Blinatumomab, Solitomab, AMG 330, MT112, MT111, BAY2010112, MEDI-565, or a mixture of any of these.

Desirable APIs and formulations of the invention may comprise antibody mimetics. Non-limiting examples of antibody mimetics which may be used according to the present invention are affibody molecules (such as ABY-025), affilins (such as SPVF 2801), affimers, affitins, alphabodies (such as CMPX-1023), anticalins, avimers, designed ankyrin repeast proteins (DARPins such as MP0112), fynomers, kunitz domain peptides (such as Ecallantide (Kalbitor)), adnectins and monobodies (such as Pegdinetanib (Angiocept)), nanoCLAMPs, single domain antibodies such as camelid antibodies, and V_{NAR} fragments obtained from IgNAR, (immunoglobulin new antigen receptor) from cartilaginous fishes, bivalent single-domain antibodies (such as caplacizumab (Cablivi)); and armadillo repeat proteins hereunder designed armadillo repeat proteins, peptide aptamers, and knottins or a mixture of any of these.

Desirable APIs and formulations of the invention may comprise human peptide hormones. Non-limiting examples of human peptide hormones which may be used according to the present invention are amylin, anti-Müllerian hormone, adiponectin, adrenocorticotropic hormone, angiotensinogen, angiotensin, antidiuretic hormone, atrial natriuretic peptide, brain natriuretic peptide, calcitonin, cholecystokinin, corticotropin-releasing hormone, cortistatin, enkephalin, endothelin, erythropoietin, follicle-stimulating hormone, galanin, gastric inhibitory polypeptide, gastrin, ghrelin, glucagon, glucagon-like peptide-1, gonadotropin-releasing hormone, growth hormone-releasing hormone, hepcidin, human chorionic gonadotropin, human placental lactogen, growth hormone, inhibin, insulin, insulin-like growth factor, leptin, lipotropin, luteinizing hormone, melanocyte stimulating hormone, motilin, orexin, osteocalcin, oxytocin, pancreatic polypeptide, parathyroid hormone, Pituitary adenylate cyclase-activating peptide, prolactin, prolactin-releasing hormone, relaxin, renin, secretin, somatostatin, growth hormone-inhibiting hormone, growth hormone release-inhibiting hormone, somatotropin release-inhibiting factor, somatotropin release-inhibiting hormone, thrombopoietin, thyroid-stimulating hormone, thyrotropin, thyrotropin-releasing hormone, vasoactive intestinal peptide, guanylin uroguanylin, tetrakosaktid, mecasermin, somapacitan, pegvisomant, vasopressin, desemopressin, terlipressin, lypressin, ornipressin, argipressin, demoxytocin, carbetocin, gonadorelin, nafarelin, histrelin, ocreotide, anreotide, vapreotide, pasireotide, ganirelix, cetrorelix, elagolix, relugolix, teriparatide, elkatonin, and the like.

Desirable APIs and formulations of the invention may comprise cytokines and analogs, including recombinant cytokines. Non-limiting examples of cytokines and analogs include IL-1 receptor antagonist, anakinra, IL-2, IL-7, IL-15, IL-21, TNF-alfa, interferon-gamma, IFN-alfa, pifonakin, mobenakin, adargileukin alfa, aldesleukin, celmoleukin, denileukin diftitox, pegaldesleukin, teceleukin, tucotuzumab celmoleukin, daniplestim, muplestim, binetrakin, atexakin alfa, emoctakin, ilodecakin, oprelvekin, edodekin alfa, cintredekin besudotox, iboctadekin, cytokines developed into protein therapeutics (e.g. bone mophogenetic protein (BMP), erythropoietin (EPO), granulocyte colony-stimulating factor (G-CSF), interferon alfa, interferon beta, IL-11, interferon gamma), and the like.

The solid cores of the formulation of the invention comprise a biologic active drug and, in this respect, may consist essentially of one or more biologic active drugs, and/or may include one or more biologic active drugs along with other excipients and/or other active ingredients.

By ‘consists essentially’ of a biologic active drug, we include that the solid core is essentially comprised only of a biologic active drug, i.e. it is free from non-biologically active substances, such as excipients, carriers and the like (vide infra), and from other active substances. This means that the core may comprise less than about 5%, such as less than about 3%, including less than about 2%, e.g. less than about 1% of such other excipients and/or active substances.

In the alternative, cores comprising the biologic active drug may include that active ingredient in admixture with one or more pharmaceutical ingredients, which may include pharmaceutically-acceptable excipients, such as adjuvants, diluents or carriers, and/or may include other biologically-active ingredients.

Non-biologically active adjuvants, diluents and carriers that may be employed in cores to be coated in accordance with the invention may include pharmaceutically-acceptable substances that are soluble in water, such as carbohydrates, e.g. sugars, such as lactose and/or trehalose, and sugar alcohols, such as mannitol, sorbitol and xylitol; or pharmaceutically-acceptable inorganic salts, such as sodium chloride. Preferred carrier/excipient materials include sugars and sugar alcohols.

The biologic active drugs may be presented in a crystalline, a part-crystalline and/or an amorphous state. The biologic active drug may be in the solid state, or may be converted into the solid state, at about room temperature (e.g. about 18° C.) and about atmospheric pressure, irrespective of the physical form. Active agent (and optionally other pharmaceutical ingredients as mentioned hereinbefore) should also remain in the form of a solid whilst being coated in, for example, an ALD reactor, and also should not decompose physically or chemically to an appreciable degree (i.e. no more than about 10% w/w) whilst being coated, or after having been covered by the coating material.

Formulations of the invention comprise a pharmacologically-effective amount of one or more biologic active drugs. Preferably, the solid cores of the formulation of the invention comprise said pharmacologically-effective amount of said one or more biologic active drugs.

The term ‘pharmacologically-effective amount’ refers to an amount of one or more biologic active drugs, which is capable of conferring a desired physiological change (such as a therapeutic effect) on a treated patient, whether administered alone or in combination with another active ingredient. Such a biological or medicinal response, or such an effect, in a patient may be subjective (i.e. the subject gives an indication of, or feels, an effect), and includes at least partial alleviation of the symptoms of the disease or disorder being treated, or curing or preventing said disease or disorder, or may be objective (i.e. measurable by some test or marker).

Dosages of one or more biologic active drugs that may be administered to a patient should thus be sufficient to affect a therapeutic response over a reasonable and/or relevant timeframe. One skilled in the art will recognize that the selection of the exact dose and composition and the most appropriate delivery regimen will also be influenced by not only the nature of the biologic active drug(s) but also inter alia the pharmacological properties of the formulation, the route of administration, the nature and severity of the condition being treated, and the physical condition and mental acuity of the recipient, as well as the age, condition, body weight, sex and response of the patient to be treated, and the stage/severity of the disease, as well as genetic differences between patients.

Dosages of one or more biologic active drugs may also be determined by the timing and frequency of administration. In any event, the medical practitioner, or other skilled person, will be able to determine routinely the actual dosage of one or more biologic active drugs, which will be most suitable for an individual patient.

When injected, formulations of the invention provide a depot formulation, from which one or more biologic active drugs are released over a prolonged period of time. That period of time may be at least about 3 days, such as about 5, or about 7, days and up to a period of about a year, such as about 3 weeks (e.g. about 2 weeks or about 4 weeks), or about 12 weeks (e.g. about 10 weeks or about 14 weeks).

Suitable doses of one or more biologic active drugs in formulations of the invention may thus provide a plasma concentration-time profile that is provides an exposure (AUC, defined as, for example AUCₗₐₛₜ (the area under plasma concentration vs. time curve up to the last detectable concentration over a prolonged period of time) or, more preferably, AUC_{∞} (the area under the plasma concentration vs. time curve up to infinite time)) that provides at least the same therapeutic effect as that obtained for current, commercial subcutaneous injections and/or intravenous infusions of one or more biologic active drugs that are used in clinical practice.

Formulations of the invention may be capable of providing an exposure, in terms of AUC_{∞}, for one or more biologic active drugs in plasma over any one of the above-mentioned time periods that is no more than 100% of the total exposure (AUC_{∞}) obtained from the current standard of care/dosing regimen administered over seven consecutive days by injection or infusions of one or more biologic active drugs.

The solid biologic active drug-containing cores of the formulation of the invention are provided in the form of nanoparticles or, more preferably, microparticles. Preferred weight-, number-, or volume-based mean diameters are between about 50 nm (e.g. about 100 nm, such as about 250 nm) and about 30 μm, for example between about 500 nm and about 100 μm, more particularly between about 1 μm and about 50 μm, such as about 25 μm, e.g. about 20 μm.

As used herein, the term ‘weight based mean diameter’ will be understood by the skilled person to include that the average particle size is characterised and defined from a particle size distribution by weight, i.e. a distribution where the existing fraction (relative amount) in each size class is defined as the weight fraction, as obtained by e.g. sieving (e.g. wet sieving). As used herein, the term ‘number based mean diameter’ will be understood by the skilled person to include that the average particle size is characterised and defined from a particle size distribution by number, i.e. a distribution where the existing fraction (relative amount) in each size class is defined as the number fraction, as measured by e.g. microscopy. As used herein, the term ‘volume based mean diameter’ will be understood by the skilled person to include that the average particle size is characterised and defined from a particle size distribution by volume, i.e. a distribution where the existing fraction (relative amount) in each size class is defined as the volume fraction, as measured by e.g. laser diffraction. The person skilled in the art will also understand there are other suitable ways of expressing mean diameters, such as area based mean diameters, and that these other expressions of mean diameter are interchangeable with those used herein. Other instruments that are well known in the field may be employed to measure particle size, such as equipment sold by e.g. Malvern Instruments, Ltd (Worcestershire, UK) and Shimadzu (Kyoto, Japan).

Particles may be spherical, that is they possess an aspect ratio smaller than about 20, more preferably less than about 10, such as less than about 4, and especially less than about 2, and/or may possess a variation in radii (measured from the centre of gravity to the particle surface) in at least about 90% of the particles that is no more than about 50% of the average value, such as no more than about 30% of that value, for example no more than about 20% of that value.

Nevertheless, the coating of particles on any shape is also possible in accordance with the invention. For example, irregular shaped (e.g. ‘raisin’-shaped), needle-shaped, flake-shaped or cuboid-shaped particles may be coated. For a non-spherical particle, the size may be indicated as the size of a corresponding spherical particle of e.g. the same weight, volume or surface area. Hollow particles, as well as particles having pores, crevices etc., such as fibrous or ‘tangled’ particles may also be coated in accordance with the invention.

Particles may be obtained in a form in which they are suitable to be coated or be obtained in that form, for example by particle size reduction processes (e.g. crushing, cutting, milling or grinding) to a specified weight based mean diameter (as hereinbefore defined), for example by wet grinding, dry grinding, air jet-milling (including cryogenic micronization), ball milling, such as planetary ball milling, as well as making use of end-runner mills, roller mills, vibration mills, hammer mills, roller mill, fluid energy mills, pin mills, etc. Alternatively, particles may be prepared directly to a suitable size and shape, for example by spray-drying, freeze-drying, spray-freeze-drying, vacuum-drying, precipitation, including the use of supercritical fluids or other top-down methods (i.e. reducing the size of large particles, by e.g. grinding, etc.), or bottom-up methods (i.e. increasing the size of small particles, by e.g. sol-gel techniques, crystallization, etc.). Nanoparticles may alternatively be made by well-known techniques, such as gas condensation, attrition, chemical precipitation, ion implantation, pyrolysis, hydrothermal synthesis, etc.

It may be necessary (depending upon how the particles that comprise the cores are initially provided) to wash and/or clean them to remove impurities that may derive from their production, and then dry them. Drying may be carried out by way of numerous techniques known to those skilled in the art, including evaporation, spray-drying, vacuum drying, freeze drying, fluidized bed drying, microwave drying, IR radiation, drum drying, etc. If dried, cores may then be deagglomerated by grinding, screening, milling and/or dry sonication. Alternatively, cores may be treated to remove any volatile materials that may be absorbed onto its surface, e.g. by exposing the particle to vacuum and/or elevated temperature.

Surfaces of cores may be chemically activated prior to applying the first layer of coating material, e.g. by treatment with hydrogen peroxide, ozone, free radical-containing reactants or by applying a plasma treatment, in order to create free oxygen radicals at the surface of the core. This in turn may produce favourable adsorption/nucleation sites on the cores for the ALD precursors.

Preferred methods of applying the coating(s) to the cores comprising biologic active drugs include gas phase techniques, such as ALD or related technologies, such as atomic layer epitaxy (ALE), molecular layer deposition (MLD; a similar technique to ALD with the difference that molecules (commonly organic molecules) are deposited in each pulse instead of atoms), molecular layer epitaxy (MLE), chemical vapor deposition (CVD), atomic layer CVD, molecular layer CVD, physical vapor deposition (PVD), sputtering PVD, reactive sputtering PVD, evaporation PVD and binary reaction sequence chemistry. ALD is the preferred method of coating according to the invention.

When ALD is employed, the coating materials may be prepared by feeding a precursor into an ALD reactor chamber (in a so called ‘precursor pulse’) to form the adsorbed atomic or molecular monolayer at the surface of the particle. A second precursor is then pulsed into the reactor and reacts with the first precursor, resulting in the formation of a monolayer of a compound on the substrate surface. A subsequent purging pulse is followed by a further pulse of the first precursor, and thus the start of a new cycle of the same events, which is an ALD cycle.

In most instances, the first of the consecutive reactions will involve some functional group or free electron pairs or radicals at the surface to be coated, such as a hydroxy group (—OH) or a primary or secondary amino group (—NH₂ or —NHR where R e.g. is an aliphatic group, such as an alkyl group). The individual reactions are advantageously carried out separately and under conditions such that all excess reagents and reaction products are essentially removed before conducting the subsequent reaction.

In ALD, layers of coating materials may be applied at process temperatures from about 20° C. to about 100° C., e.g. from about 40° C. to about 100°. The optimal process temperature depends on the reactivity of the precursors. It is preferred that a lower temperature, such as from about 30° C. to about 100° C. is employed. In particular, a temperature from about 20° C. to about 80° C. is employed, such as from about 30° C. to about 70° C., such as from about 40° C. to about 60° C., such as about 50° C.

The cores may be coated with one or more separate, discrete layers, of inorganic coatings as defined herein. Preferably, more than one separate, discrete inorganic layers, coatings or shells (which terms are used herein interchangeably) are applied (that is ‘separately applied’) to the solid cores comprising a biologic active drug sequentially.

By ‘separate application’ of ‘separate layers, coatings or shells’, we mean that the solid cores are coated with a first layer of coating material, which layer is formed by more than one (e.g. a plurality or a set of) cycles as described herein, each cycle producing a monolayer of inorganic coating material, and then that resultant coated core is subjected to some form of deagglomeration process.

In other words, ‘gas-phase deposition (e.g. ALD) cycles’ can be repeated several times to provide a ‘gas-phase deposition (e.g. ALD) set’ of cycles, which may consist of e.g. 10, 25 or 100 cycles. However, after this set of cycles, the coated core is subjected to some form of deagglomeration process, which is followed by a further set of cycles.

This process may be repeated as many times as is desired and, accordingly, the number of discrete layers of coating material(s) produced by sets of cycles that is in a final coating corresponds to the number of these intermittent deagglomeration steps with a final mechanical deagglomeration being conducted prior to the application of a final layer (set of cycles) of coating material.

The terms ‘disaggregation’ and ‘deagglomeration’ are used interchangeably when referring to the coated particles, and disaggregating coated particles aggregates is preferably done by way of a mechanical sieving technique.

Coated cores may be subjected to the aforementioned deagglomeration process internally, without being removed from said apparatus by way of a continuous process. Such a process will involve forcing solid product mass formed by coating said cores through a sieve that is located within the reactor, and is configured to deagglomerate any particle aggregates upon forcing of the coated cores by means of a forcing means applied within said reactor, prior to being subjected to a second and/or a further coating. This process is continued for as many times as is required and/or appropriate prior to the application of the final coating as described herein.

Having the sieve located within the reactor vessel means that the coating can be applied by way of a continuous process which does not require the particles to be removed from the reactor. Thus, no manual handling of the particles is required, and no external machinery is required to deagglomerate the aggregated particles. This not only considerably reduces the time of the coating process being carried out, but is also more convenient and reduces the risk of harmful (e.g. poisonous) materials being handled by personnel. It also enhances the reproducibility of the process by limiting the manual labour and reduces the risk of contamination.

Alternatively, and/or preferably, coated cores may be removed from the coating apparatus, such as the ALD reactor, and thereafter subjected to an external deagglomeration step, for example as described in international patent application WO 2014/187995. Such an external deagglomeration step may comprise agitation, such as sonication in the wet or dry state, or preferably may comprise subjecting the resultant solid product mass that has been discharged from the reactor to sieving, e.g. by forcing it through a sieve or mesh in order to deagglomerate the particles, for example as described hereinafter, prior to placing the particles back into the coating apparatus for the next coating step. Again, this process may be continued for as many times as is required and/or appropriate prior to the application of the final coating.

In an external deagglomeration process, deagglomeration may alternatively be effected (additionally and/or instead of the abovementioned processes) by way of subjecting the coated particles in the wet or dry state to one or more of nozzle aerosol generation, milling, grinding, stirring, high sheer mixing and/or homogenization. If the step(s) of deagglomeration are carried out on particles in the wet state, the deagglomerated particles should be dried (as hereinbefore described in relation to cores) prior to the next coating step.

However, we prefer that, in such an external process, the deagglomeration step(s) comprise one or more sieving step(s), which may comprise jet sieving, manual sieving, vibratory sieve shaking, horizontal sieve shaking, tap sieving, or (preferably) sonic sifting as described hereinafter, or a like process, including any combination of these sieving steps. Manufacturers of suitable sonic sifters include Advantech Manufacturing, Endecott and Tsutsui.

Vibrational sieving techniques may involve a means of vibrationally forcing the solid product mass formed by coating said cores through a sieve that is located internally or (preferably) externally to (i.e. outside of) the reactor, and is configured to deagglomerate any particle aggregates upon said vibrational forcing of the coated cores, prior to being subjected to a second and/or a further layer of coating material. This process is repeated as many times as is required and/or appropriate prior to the application of a final layer of coating material.

Vibrational forcing means may comprise a vibration motor which is coupled to a sieve. The vibration motor is configured to vibrate and/or gyrate when an electrical power is supplied to it. For example, the vibration motor may be a piezoelectric vibration motor comprising a piezoelectric material which changes shape when an electric field is applied, as a consequence of the converse piezoelectric effect. The changes in shape of the piezoelectric material cause acoustic or ultrasonic vibrations of the piezoelectric vibration motor.

The vibration motor may alternatively be an eccentric rotating mass (ERM) vibration motor comprising a mass which is rotated when electrical power is supplied to the motor. The mass is eccentric from the axis of rotation, causing the motor to be unbalanced and vibrate and/or gyrate due to the rotation of the mass. Further, the ERM vibration motor may comprise a plurality of masses positioned at different locations relative to the motor. For example, the ERM vibration motor may comprise a top mass and a bottom mass each positioned at opposite ends of the motor. By varying each mass and its angle relative to the other mass, the vibrations and/or gyrations of the ERM vibration motor can be varied.

The vibration motor is coupled to the sieve in a manner in which vibrations and/or gyrations of the motor when electrical power is supplied to it are transferred to the sieve.

The sieve and the vibration motor may be suspended from a mount (such as a frame positionable on a floor, for example) via a suspension means such that the sieve and motor are free to vibrate relative to the mount without the vibrations being substantially transferred to or dampened by the mount. This allows the vibration motor and sieve to vibrate and/or gyrate without impediment and also reduces noise generated during the vibrational sieving process. The suspension means may comprise one or more springs or bellows (i.e. air cushion or equivalent cushioning means) that couple the sieve and/or motor to the mount. Manufacturers of vibratory sieves or sifters suitable for carrying out such a process include for instance Russell Finex, SWECO, Filtra Vibracion, VibraScreener, Gough Engineering and Farley Greene.

Preferably, the vibrational sieving technique further comprises controlling a vibration probe coupled to the sieve. The vibration probe may be controlled to cause the sieve to vibrate at a separate frequency to the frequency of vibrations caused by the vibration motor. Preferably the vibration probe causes the sieve to vibrate at a higher frequency than the vibrations caused by the vibration motor and, more preferably, the frequency is within the ultrasonic range.

Providing additional vibrations to the sieve by means of the vibration probe reduces the occurrence of clogging in the sieve, reduces the likelihood of the sieve being overloaded and decreases the amount of time needed to clean the mesh of the sieve.

Preferably, the aforesaid vibrational sieving technique comprises sieving coated particles with a throughput of at least 1 g/minute. More preferably, the vibrational sieving technique comprises sieving coated particles with a throughput of 4 g/minute or more.

The throughput depends on the area of the sieve mesh, mesh-size of the sieve, the particle size, the stickiness of the particles, static nature of the particle. By combining some of these features a much higher throughput is possible. Accordingly, the vibrational sieving technique may more preferably comprise sieving coated particles with a throughput of up to 1 kg/minute or even higher.

Any one of the above-stated throughputs represents a significant improvement over the use of known mechanical sieving, or sifting, techniques. For example, we found that sonic sifting involved sifting in periods of 15 minutes with a 15-minute cooling time in-between, which is necessary for preserving the apparatus. To sift 20 g of coated particles required 9 sets of 15 minutes of active sifting time, i.e. a total time (including the cooling) of 255 minutes. By comparison, by using the aforementioned vibrational sieving technique, 20 g of coated particles may be sieved continuously in, at most, 20 minutes, or more preferably in just 5 minutes, or less.

The sieve mesh size may be determined so that the ratio of the size of the sieved or sonic sifted particles to the sieve mesh size is about 1:>1, preferably about 1:2, and optionally about 1:4. The size mesh size may range from about 20 μm to about 100 μm, preferably from about 20 μm to about 60 μm.

Appropriate sieve meshes may include perforated plates, microplates, grid, diamond, threads, polymers or wires (woven wire sieves) but are preferably formed from metals, such as stainless steel.

Surprisingly, using a stainless steel mesh within the vibrational sieving technique is as gentle to the particle coatings as using a softer polymer sieve as part of a mechanical sieving technique such as sonic sifting.

Also, a known problem with sieving powders is the potentially dangerous generation of static electricity. A steel mesh has the advantage of removing static electricity from the powder while that is not the case with a polymeric mesh, which has to be used in a sonic sifter.

Further, the mesh size of known sonic sifters is limited to about 100 μm since the soundwaves travel through the mesh rather than vibrating it. That limitation does not exist using for vibrational sieving techniques as there is no reliance on soundwaves to generate vibrations in the sieve. Therefore, the vibrational sieving technique described herein allows larger particles to be sieved than if alternative mechanical sieving techniques were used.

If a (e.g. vibrational) sieve is located externally to (i.e. outside of) the reactor, the process for making coated cores of formulations of the invention comprises discharging the coated particles from the gas phase deposition reactor prior to subjecting the coated particles to agitation, followed by reintroducing the deagglomerated, coated particles into the gas phase deposition reactor prior to applying a further layer of at least one coating material to the reintroduced particles.

We have found that applying separate layers of coating materials following external deagglomeration gives rise to visible and discernible interfaces that may be observed by analysing coated particles according to the invention, and are observed by e.g. TEM as regions of higher electron permeability. In this respect, the thickness of the layers between interfaces correspond directly to the number of cycles in each series that are carried out within the ALD reactor, and between individual external agitation steps.

Because, in an ALD coating process, coating takes place at the atomic level, such clear, physical interfaces are typically more difficult to observe.

Without being limited by theory, it is believed that removing coated particles from the vacuum conditions of the ALD reactor and exposing a newly-coated surface to the atmosphere results in structural rearrangements due to relaxation and reconstruction of the outermost atomic layers. Such a process is believed to involve rearrangement of surface (and near surface) atoms, driven by a thermodynamic tendency to reduce surface free energy.

Furthermore, surface adsorption of species, e.g. hydrocarbons that are always present in the air, may contribute to this phenomenon, as can surface modifications, due to reaction of coatings formed with hydrocarbons, as well as atmospheric oxygen and the like. Accordingly, if such interfaces are analysed chemically, they may contain traces of contaminants or the core material, such as an API forming part of the core, that do not originate from the coating process, such as ALD.

Whether carried out inside or outside of the reactor, particle aggregates are preferably broken up by a forcing means that forces them through a sieve, thus separating the aggregates into individual particles or aggregates of a desired and predetermined size (and thereby achieving deagglomeration). In the latter regard, in some cases the individual primary particle size is so small (i.e. <1 μm) that achieving ‘full’ deagglomeration (i.e. where aggregates are broken down into individual particles) is not possible. Instead, deagglomeration is achieved by breaking down larger aggregates into smaller aggregates of secondary particles of a desired size, as dictated by the size of the sieve mesh. The smaller aggregates are then coated by the gas phase technique to form fully coated ‘particles’ in the form of small aggregate particles. In this way, the term ‘particles’, when referring to the particles that have been deagglomerated and coated in the context of the invention, refers to both individual (primary) particles and aggregate (secondary) particles of a desired size.

In any event, the desired particle size (whether that be of individual particles or aggregates of a desired size) is maintained and, moreover, continued application of the gas phase coating mechanism to the particles after such deagglomeration via the sieving means that a complete coating is formed on the particle, thus forming fully coated particles (individual or aggregates of a desired size).

Whether carried out inside or outside of the reactor, the above-described repeated coating and deagglomeration process may be carried out at least 1, preferably 2, more preferably 3, such as 4, including 5, more particularly 6, e.g. 7 times, and no more than about 100 times, for example no more than about 50 times, such as no more than about 40 times, including no more than about 30 times, such as between 2 and times, e.g. between 3 and 15 times, such as 10 times, e.g. 9 or 8 times, more preferably 6 or 7 times, and particularly 4 or 5 times.

Whether carried out inside or outside of the reactor, it is preferred that at least one sieving step is carried out and further that that step preferably comprises a vibrational sieving step as described above. It is further preferred that at least the final sieving step comprises a vibrational sieving step being conducted prior to the application of a final layer (set of cycles) of coating material. However, it is further preferred that more than one (including each) of the sieving steps comprise vibrational sieving techniques, steps or processes as described herein.

The preferable repetition of these steps makes the improved throughput of any vibrational sieving technique all the more beneficial.

The total thickness of the coating (meaning all the separate layers/coatings/shells) will on average be in the region of between about 0.5 nm and about 2 μm.

The minimum thickness of each individual layer/coating/shell will on average be in the region of about 0.1 nm (including about 0.5 nm, for example about 0.75 nm, such as about 1 nm).

The maximum thickness of each individual layer/coating/shell will depend on the size of the core (to begin with), and thereafter the size of the core with the coatings that have previously been applied, and may be on average about 1 hundredth of the mean diameter (i.e. the weight-, number-, or volume-based mean diameter) of that core, or core with previously-applied coatings.

Preferably, for particles with a mean diameter that is between about 100 nm and about 1 μm, the total coating thickness should be on average between about 1 nm and about nm; for particles with a mean diameter that is between about 1 μm and about 20 μm, the coating thickness should be on average between about 1 nm and about 10 nm; for particles with a mean diameter that is between about 20 μm and about 700 μm, the coating thickness should be on average between about 1 nm and about 100 nm.

We have found that applying coatings/shells followed by conducting one or more deagglomeration step such as sonication gives rise to abrasions, pinholes, breaks, gaps, cracks and/or voids (hereinafter ‘cracks’) in the layers/coatings, due to coated particles essentially being more tightly ‘bonded’ or ‘glued’ together directly after the application of a thicker coating. This may expose a core comprising biologically-active ingredient to the elements once deagglomeration takes place.

As it is intended to provide particles in an aqueous suspension prior to administration to a patient, it is necessary to provide deagglomerated primary particles without pinholes or cracks in the coatings. Such cracks will result in an undesirable initial peak (burst) in plasma concentration of active ingredient directly after administration.

We have found that, by conducting one or more of the deagglomeration steps described herein, this gives rise to significantly less pinholes, gaps or cracks in the final layer of coating material, giving rise to particles that are not only completely covered by that layer/coating, but are also covered in a manner that enables the particles to be deagglomerated readily (e.g. using a non-aggressive technique, such as vortexing) in a manner that does not destroy the layers of coating material that have been formed, prior to, and/or during, pharmaceutical formulation.

In this respect, the coating of (e.g. inorganic) material typically completely surrounds, encloses and/or encapsulates said solid cores comprising biologic active drug(s). In this way, the risk of an initial drug concentration burst due to the drug coming into direct contact with solvents in which the relevant active ingredient is soluble is minimized. This may include not only bodily fluids, but also any medium in which such coated particles may be suspended prior to injection.

Thus in a further embodiment of the invention, there are provided particles as hereinbefore disclosed, wherein said coating surrounding, enclosing and/or encapsulating said core covers at least about 50%, such as at least about 65%, including at least about 75%, such as at least about 80%, more particularly at least about 90%, such as at least about 91%, such as at least about 92%, such as at least about 93%, such as at least about 94%, such as at least about 95%, such as at least about 96%, such as at least about 97%, such as at least about 98%, such as at least about 99%, such as approximately, or about, 100%, of the surface of the solid core, such that the coating essentially completely surrounds, encloses and/or encapsulates said core.

As used herein, the term ‘essentially completely coating completely surrounds, encloses and/or encapsulates said core’ means a covering of at least about 98%, or at least about 99%, of the surface of the solid core.

In the alternative, processes described herein may result in the deagglomerated coated particles with the essential absence of said cracks through which active ingredient can be released in an uncontrolled way.

Although some minor cracks may appear in the said coating without effecting the essential function thereof in terms of controlling release, in a further embodiment, there are provided particles as hereinbefore disclosed, wherein at least about 90% of the particles do not exhibit cracks in the coating surrounding, enclosing and/or encapsulating said core. In one embodiment at least about 91%, such as at least about 92%, such as at least about 93%, such as at least about 94%, such as at least about 95%, such as at least about 96%, such as at least about 97%, such as at least about 98%, such as at least about 99%, such as approximately 100% of the particles do not exhibit said cracks.

Alternatively, by ‘essentially free of said cracks’ in the coating(s), we also mean that less than about 1% of the surfaces of the coated particles comprise abrasions, pinholes, breaks, gaps, cracks and/or voids through which active ingredient is potentially exposed (to, for example, the elements).

The layers of coating material may, taken together, be of an essentially uniform thickness over the surface area of the particles. By ‘essentially uniform’ thickness, we mean that the degree of variation in the thickness of the coating of at least about 10%, such as about 25%, e.g. about 50%, of the coated particles that are present in a formulation of the invention, as measured by TEM, is no more than about ±20%, including ±50% of the average thickness.

Inorganic coating materials may comprise one or more metals or metalloids, or may comprise one or more metal-containing, or metalloid-containing, compounds, such as metal, or metalloid, oxides, nitrides, sulphides, selenides, carbonates, and/or other ternary compounds, etc. Metal, and metalloid, hydroxides and, especially, oxides are preferred, especially metal oxides.

Metals that may be mentioned include alkali metals, alkaline earth metals, noble metals, transition metals, post-transition metals, lanthanides, etc. Metal and metalloids that may be mentioned include aluminium, titanium, magnesium, iron, gallium, zinc, zirconium, niobium, hafnium, tantalum, lanthanum, and/or silicon; more preferably aluminium, titanium, magnesium, iron, gallium, zinc, zirconium, and/or silicon; especially aluminium, silicon, titanium and/or zinc.

As mentioned above, as the compositions made by the process of the invention comprises two or more discrete layers of inorganic coating materials, the nature and chemical composition(s) of those layers may differ from layer to layer.

Individual layers may also comprise a mixture of two or more inorganic materials, such as metal oxides or metalloid oxides, and/or may comprise multiple layers or composites of different inorganic or organic materials, to modify the properties of the layer.

Coating materials that may be mentioned include those comprising aluminium oxide (Al₂O₃), titanium dioxide (TiO₂), iron oxides (FeₓO_{y}, e.g. FeO and/or Fe₂O₃ and/or Fe₃O₄), gallium oxide (Ga₂O₃), magnesium oxide (MgO), zinc oxide (ZnO), niobium oxide (Nb₂O₅), hafnium oxide (HfO₂), tantalum oxide (Ta₂O₅), lanthanum oxide (La₂O₃), zirconium dioxide (ZrO₂) and/or silicon dioxide (SiO₂). Preferred coating materials include aluminium oxide, titanium dioxide, iron oxides, gallium oxide, magnesium oxide, zinc oxide, zirconium dioxide and silicon dioxide. More preferred coating materials include iron oxide, titanium dioxide, zinc sulphide, more preferably zinc oxide, silicon dioxide and/or aluminium oxide.

Layers of coating materials (on an individual or a collective basis) may consist essentially (e.g. is greater than about 80%, such as greater than about, 90%, e.g. about 95%, such as about 98%) of iron oxides, titanium dioxide, or more preferably zinc oxide, silicon oxide and/or aluminium oxide.

There is thus further provided a method of preparing of plurality of coated particles in accordance with the invention, wherein the coated particles are made by applying precursors of at least two metal and/or metalloid oxides forming a mixed oxide on the solid cores, and/or previously-coated solid cores, by a gas phase deposition technique. Precursors for forming a metal oxide or a metalloid oxide often include an oxygen precursor, such as water, oxygen, ozone and/or hydrogen peroxide; and a metal and/or metalloid compound, typically an organometal compound or an organometalloid compound.

Non-limiting examples of precursors are as follows: Precursors for zinc oxide may be water and diC₁-C₅alkylzinc, such as diethylzinc. Precursors for aluminium oxide may be water and triC₁-C₅alkylaluminium, such as trimethylaluminium. Precursors for silicon oxide (silica) may be water as the oxygen precursor and silanes, alkylsilanes, aminosilanes, and orthosilicic acid tetraethyl ester. Precursors for iron oxide includes oxygen, ozone and water as the oxygen precursor; and di C₁-C₅alkyl-iron, dicyclopropyl-iron, and FeCl₃. It will be appreciated that the person skilled in the art is aware of what precursors are suitable for the purpose as disclosed herein.

It is further preferred that the inorganic coating material comprising mixture of:
(i) zinc oxide (ZnO); and
(ii) one or more other metal and/or metalloid oxides,
   wherein the atomic ratio ((i):(ii)) is between at least about 1:6 and up to and including about 6:1.

Preferably, the atomic ratio ((i):(ii)) is between at least about 1:1 and up to and including about 6:1.

The coating of comprising a mixture of zinc oxide and one or more other metal and/or metalloid oxides is referred to hereinafter as a ‘mixed oxide’ coating or coating material(s).

The biologic active drug-containing cores may thus be coated with a coating material that comprises a mixture of zinc oxide, and one or more other metal and/or metalloid oxides, at an atomic ratio of zinc oxide to the other oxide(s) that is at least about 1:6 (e.g. at least about 1:4, such as at least 1:2), preferably at least about 1:1 (e.g. at least about 1.5:1, such as at least about 2:1), including at least about 2.25:1, such as at least about 2.5:1 (e.g. at least about 3.25:1 or least about 2.75:1 (including 3:1)), and is up to (i.e. no more than) and including about 6:1, including up to about 5.5:1, or up to about 5:1, such as up to about 4.5:1, including up to about 4:1 (e.g. up to about 3.75:1).

In order to make a mixed oxide coating with an atomic ratio of (for example) between about 1:1 and up to and including about 6:1 of zinc oxide relative to the one or more other metal and/or metalloid oxides, the skilled person will appreciate that for every one ALD cycle (i.e. monolayer) of the other oxide(s), between about 1 and about 6 ALD cycles of zinc oxide must also be deposited. For example, for a 3:1 atomic (zinc:other oxide) mixed oxide coating to be formed, 3 zinc-containing precursor pulses may each be followed by second precursor pulses, forming 3 monolayers of zinc oxide, which will then be followed by 1 pulse of the other metal and/or metalloid-containing precursor followed by second precursor pulse, forming 1 monolayer of oxide of the other metal and/or metalloid. Alternatively, 6 monolayers of zinc oxide may be followed by 2 monolayers of the other oxide, or any other combination so as to provide an overall atomic ratio of about 3:1. In this respect, the order of pulses to produce the relevant oxides is not critical, provided that the resultant atomic ratio is in the relevant range in the end.

We have found that, when coatings comprising zinc oxide are applied using ALD at a lower temperature, such as from about 50° C. to about 100° C. (unlike other coating materials, such as aluminium oxide and titanium oxide, which form amorphous layers) the coating materials are largely crystalline in their nature.

Without being limited by theory, because zinc oxide is crystalline, if only zinc oxide is employed as coating material, we are of the understanding that interfaces may be formed between adjacent crystals of zinc oxide that are deposited by ALD, through which a carrier system, medium or solvent in which zinc oxide is partially soluble (e.g. an aqueous solvent system) can ingress following suspension therein. It is believed that this may give rise to dissolution that is too fast for the depot-forming composition that it is intended to make.

We have now found that these problems may be alleviated by making a mixed oxide coating as described herein. In particular, we have now found that these problems may be alleviated by making a mixture of two or more metal and/or metalloid oxides (mixed oxide) coating as described herein. In particular, by forming a mixed oxide coating as described herein, that may be predominantly, but not entirely, comprised of zinc oxide, we have been able to coat active ingredients with coatings that appear to be essentially amorphous, or a composite between crystalline and amorphous material, and/or in which ingress of injection vehicles such as water may be reduced. In this respect, it appears to us that the presence of the aforementioned perceived interfaces may be reduced, or avoided altogether, by employing the mixed oxide aspect of the invention, in either a heterogeneous manner (in which the other oxide is ‘filling in’ gaps formed by the interfaces), or in a homogeneous manner (in which a true composite of mixed oxide materials is formed during deposition, in a manner where the interfaces are potentially avoided in the first place).

In addition to the inorganic coating that is employed in formulations of the invention, other coating materials, which may be pharmaceutically-acceptable and essentially non-toxic coating materials may also be applied in addition, either between separate inorganic coatings (e.g. in-between separate deagglomeration steps) and/or whilst an inorganic coating is being applied herein. Such materials may comprise multiple layers or composites of coating materials as defined herein and one or more different inorganic or organic materials, to modify the properties of the layer(s).

Additional coating materials may comprise organic or polymeric materials, such as a polyamide, a polyimide, a polyurea, a polyurethane, a polythiourea, a polyester or a polyimine. Additional coating materials may also comprise hybrid materials (as between organic and inorganic materials), including materials that are a combination between a metal, or another element, and an alcohol, a carboxylic acid, an amine or a nitrile. However, we prefer that coating materials comprise inorganic materials.

The gas phase deposition reactor chamber used may optionally, and/or preferably, b a stationary gas phase deposition reactor chamber. The term ‘stationary’, in the context of gas phase deposition reactor chambers, will be understood to mean that the reactor chamber remains stationary while in use to perform a gas phase deposition technique, excluding negligible movements and/or vibrations such as those caused by associated machinery for example.

Additionally, a so-called ‘stop-flow’ process may be employed. Using a stop-flow process, once the first precursor has been fed into the reactor chamber and prior to the first precursor being purged from the reactor chamber, the first precursor may be allowed to contact the cores in the reactor chamber for a pre-determined period of time (which may be considered as a soaking time). During the pre-determined period of time there is preferably a substantial absence of pumping that may result in flow of gases and/or a substantial absence of mechanical agitation of the cores.

The employment of the stop-flow process may increase coating uniformity by allowing each gas to diffuse conformally in high aspect-ratio substrates, such as powders. The benefits may be even more pronounced when using precursors with slow reactivity as more time is given for the precursor to react on the surface. This may be evident especially when depositing mixed oxide coatings according to the invention. For example, when depositing a mixed zinc oxide/aluminium oxide coating as described hereinafter, we have found that a zinc-containing precursor, such as diethylzinc (DEZ), which has a lower reaction probability towards the surface of a substrate than, for example, aluminium containing precursors, such as trimethylaluminum (TMA).

In addition to generating coatings with good shell integrity and more controlled release profiles, the employment of such a stop-flow process may improve the ability to achieve a particular coating composition.

For example, when attempting to employ a gas phase technique to produce a coating comprising an atomic ratio of 3:1 between zinc and aluminium in the resulting shell as described above, we have found that a ratio that is much closed to 3:1 may be achieved using a stop-flow process than when depositing material using a continuous flow of precursors.

Preferably, and/or optionally, a ‘multi-pulse’ technique may also be employed to feed the first precursor, the second precursor or both precursors to the reactor chamber.

Using such a multi-pulse technique, the respective precursor may be fed into the reactor chamber as a plurality of ‘sub-pulses’, each lasting a short period of time such as 1 second up to about a minute (depending on the size and the nature of the gas phase deposition reactor), rather than as one continuous pulse. The precursor may be allowed to contact the cores in the reactor chamber for the pre-determined period of time, for example from about 1 to 500 seconds, about 2 to 250 seconds, about 3 to 100 seconds, about 4 to 50 seconds, or about 5 to 10 seconds, for example 9 seconds, after each sub-pulse. Again, depending on the size and the nature of the gas phase deposition reactor, this time could be extended up to several minutes (e.g. up to about 30 minutes). The introduction of a sub-pulse followed by a period of soaking time may be repeated a pre-determined number of times, such as between about 5 to 1000 times, about 10 to 250 times, or about 20 to 50 times in a single step.

Although the plurality of coated particles in accordance with the invention are essentially free of the aforementioned cracks in the applied coatings, through which active ingredient is potentially exposed (to, for example, the elements), two further, optional steps may be applied to the plurality of coated particles prior to subjecting it to further pharmaceutical formulation processing.

The first optional step may comprise, subsequent to the final deagglomeration step as hereinbefore described, application of a final overcoating layer, the thickness of which outer ‘overcoating’ layer/coating, or ‘sealing shell’ (which terms are used herein interchangeably), must be thinner than the previously-applied separate layers/coatings/shells (or ‘subshells’).

The thickness may therefore be on average no more than a factor of about 0.7 (e.g. about 0.6) of the thickness of the widest previously-applied subshell. Alternatively, the thickness may be on average no more than a factor of about 0.7 (e.g. about 0.6) of the thickness of the last subshell that is applied, and/or may be on average no more than a factor of about 0.7 (e.g. about 0.6) of the average thickness of all of the previously-applied subshells. The thickness may be on average in the region of about 0.3 nm to about 10 nm, for particles up to about 20 μm. For larger particles, the thickness may be on average no more than about 1/1000 of the coated particles' weight-, number-, or volume-based mean diameter.

The role of such as sealing shell is to provide a ‘sealing’ overcoating layer on the particles, covering over those cracks, so giving rise to particles that are not only completely covered by that sealing shell, but also covered in a manner that enables the particles to be deagglomerated readily (e.g. using a non-aggressive technique, such as vortexing) in a manner that does not destroy the subshells that have been formed underneath, prior to, and/or during, pharmaceutical formulation.

For the reasons described herein, it is preferred that the sealing shell does not comprise zinc oxide. The sealing shell may on the other hand comprise silicon dioxide or, more preferably, aluminium oxide.

The second optional step may comprise ensuring that the few remaining particles with broken and/or cracked shells/coatings are subjected to a treatment in which all particles are suspended in a solvent in which the biologic active drug is soluble (e.g. with a solubility of at least about 0.1 mg/mL), but the least soluble material in the inorganic coating is insoluble (e.g. with a solubility of no more than about 0.1 μg/mL), followed by separating solid matter particles from solvent by, for example, centrifugation, sedimentation, flocculation and/or filtration, resulting in mainly intact particles being left.

The above-mentioned optional step provides a means of potentially reducing further the likelihood of a (possibly) undesirable initial peak (burst) in plasma concentration of active ingredient, as discussed hereinbefore.

At the end of the process, coated particles may be dried using one or more of the techniques that are described hereinbefore for drying cores. Drying may take place in the absence, or in the presence, of one or more pharmaceutically-acceptable excipients (e.g. a sugar or a sugar alcohol).

Alternatively, at the end of the process, separated particles may be resuspended in a solvent (e.g. water, with or without the presence of one or more pharmaceutically acceptable excipients as defined herein), for subsequent storage and/or administration to patients.

Prior to applying the first layer of coating material or between successive coatings, cores and/or partially coated particles may be subjected to one or more alternative and/or preparatory surface treatments. In this respect, one or more intermediary layers comprising different materials (i.e. other than the inorganic material(s)) may be applied to the relevant surface, e.g. to protect the cores or partially-coated particles from unwanted reactions with precursors during the coating step(s)/deposition treatment, to enhance coating efficiency, or to reduce agglomeration.

An intermediary layer may, for example, comprise one or more surfactants, with a view to reducing agglomeration of particles to be coated and to provide a hydrophilic surface suitable for subsequent coatings. Suitable surfactants in this regard include well known non-ionic, anionic, cationic or zwitterionic surfactants, such as the Tween series, e.g. Tween 80. Alternatively, cores may be subjected to a preparatory surface treatment if the active ingredient that is employed as part of (or as) that core is susceptible to reaction with one or more precursor compounds that may be present in the gas phase during the coating (e.g. the ALD) process.

Application of ‘intermediary’ layers/surface treatments of this nature may alternatively be achieved by way of a liquid phase non-coating technique, followed by a lyophilisation, spray drying or other drying method, to provide particles with surface layers to which coating materials may be subsequently applied.

Outer surfaces of particles of formulations of the invention may also be derivatized or functionalized, e.g. by attachment of one or more chemical compounds or moieties to the outer surfaces of the final layer of coating material, e.g. with a compound or moiety that enhances the targeted delivery of the particles within a patient to whom the nanoparticles are administered. Such a compound may be an organic molecule (such as PEG) polymer, an antibody or antibody fragment, or a receptor-binding protein or peptide, etc.

Alternatively, the moiety may be an anchoring group such as a moiety comprising a silane function (see, for example, Herrera et al., J. Mater. Chem., 18, 3650 (2008) and U.S. Pat. No. 8,097,742). Another compound, e.g. a desired targeting compound may be attached to such an anchoring group by way of covalent bonding, or non-covalent bonding, including hydrogen bonding, or van der Waals bonding, or a combination thereof.

The presence of such anchoring groups may provide a versatile tool for targeted delivery to specific sites in the body. Alternatively, the use of compounds such as PEG may cause particles to circulate for a longer duration in the blood stream, ensuring that they do not become accumulated in the liver or the spleen (the natural mechanism by which the body eliminates particles, which may prevent delivery to diseased tissue).

Cores coated with an inorganic coating, whether in the form of separate, discrete layers, coatings or shells or otherwise, as defined herein are referred to hereinafter as ‘the coated particles of the formulation of the invention’.

Pharmaceutical (or veterinary) formulations of the invention may include particles of different types, for example particles comprising different functionalization (as described hereinbefore), particles of different sizes, and/or different thicknesses of the layers of inorganic coating materials, or a combination thereof. By combining, in a single pharmaceutical formulation, particles with different coating thicknesses and/or different core sizes, the drug release following administration to patient may be controlled (e.g. varied or extended) over a specific time period.

Formulations of the invention may be administered systemically, for example by injection or infusion, intravenously or intraarterially (including by intravascular or other perivascular devices/dosage forms (e.g. stents)), intramuscularly, intraosseously, intracerebrally, intracerebroventricularly, intrasynovially, intrasternally, intrathecally, intralesionally, intracranially, intratumorally, cutaneously, intracutaneous, subcutaneously, transdermally, in the form of a pharmaceutically-(or veterinarily) acceptable dosage form.

The preparation of formulation of the invention comprises incorporation of coated particles as described herein into an appropriate pharmaceutically-acceptable aqueous carrier system, and may be achieved with due regard to the intended route of administration and standard pharmaceutical practice. Thus, appropriate excipients should be chemically inert to the active agent that is employed, and have no detrimental side effects or toxicity under the conditions of use. Such pharmaceutically-acceptable carriers may also impart an immediate, or a modified, release of the biologic active agent from the particles of the formulations of the invention.

For parenteral administration, such as subcutaneous and/or intramuscular injections, the compositions made by the process of the invention may be in the form of sterile injectable and/or infusible dosage forms, for example, sterile oleaginous or, preferably, aqueous suspensions of compositions made by the process of the invention.

Sterile aqueous suspensions of the particles of the formulation of the invention may be formulated according to techniques known in the art. The aqueous media should contain at least about 50% water, but may also comprise other aqueous excipients, such as Ringer's solution, and may also include polar co-solvents (e.g. ethanol, glycerol, propylene glycol, 1,3-butanediol, polyethylene glycols of various molecular weights and tetraglycol); viscosity-increasing, or thickening, agents (e.g. carboxymethylcellulose, microcrystalline cellulose, hydroxypropylmethyl cellulose, hydroxyethyl cellulose, ethyl hydroxyethyl cellulose, sodium starch glycolate, Poloxamers, such as Poloxamer 407, polyvinylpyrrolidone, cyclodextrins, such as hydroxypropyl-β-cyclodextrin, polyvinylpyrrolidone and polyethylene glycols of various molecular weights); surfactant/wetting agents to achieve a homogenous suspension (e.g. sorbitan esters, sodium lauryl sulfate; monoglycerides, polyoxyethylene esters, polyoxyethylene alkyl ethers, polyoxylglycerides and, preferably, Tweens (Polysorbates), such as Tween 80 and Tween 20). Preferred ingredients include isotonicity-modifying agents (e.g. sodium lactate, dextrose and, especially, sodium chloride); pH adjusting and/or buffering agents (e.g. citric acid, sodium citrate, and especially phosphate buffers, such as disodium hydrogen phosphate dihydrate, sodium acid phosphate, sodium dihydrogen phosphate monohydrate, and combinations thereof, which may be employed in combination with standard inorganic acids and bases, such as hydrochloric acid and sodium hydroxide); as well as other ingredients, such as mannitol, croscarmellose sodium and hyaluronic acid.

Oleaginous, or oil-based carrier systems may comprise one or more pharmaceutically- or veterinarily-acceptable liquid lipid, which may include fixed oils, such as mono-, di- or triglycerides, including miglyol (e.g. 812N), propylene glycol dicaprylocaprate (Miglyol 840, C₈/C₁₀ esters), tricaprylin (Miglyol oil), gelucire 43/01, kollisolv GTA, labrafil. The carrier systems may also comprise polysorbates, such as polysorbate 20, polysorbate 60, polysorbate 80, glycols, such as propylene glycol, polyethylene glycol, polyethylene glycol 300, polyethylene glycol 400, polyethylene glycol 600, and/or natural and/or refined pharmaceutically-acceptable oils, such as olive oil, peanut oil, soybean oil, corn oil, cottonseed oil, sesame oil, castor oil, oleic acid, and their polyoxyethylated versions (e.g. sorbitan trioleate, lauroglycol 90, capryol PGMC, PEG-60 hydrogenated castor oil, polyoxyl 35 castor oil). More preferred carrier systems include mono-, di- and/or triglycerides, wherein most preferred is medium chain triglycerides, such as alkyl chain triglycerides (e.g. C₆-C₁₂ alkyl chain triglycerides).

Such injectable suspensions may be formulated in accordance with techniques that are well known to those skilled in the art, by employing suitable dispersing or wetting agents (e.g. Tweens, such as Tween 80), and suspending agents.

Formulations of the invention may further be formulated in the form of injectable suspension of coated particles with a size distribution that is both even and capable of forming a stable suspension within the injection liquid (i.e. without settling) and may be injected through a needle. In this respect, the formulations of the invention may comprise an aqueous medium that comprises inactive ingredients that may prevent premature gelling of the formulations of the invention, and or is viscous enough to prevent sedimentation, leading to suspensions that are not ‘homogeneous’ and thus the risk of under or overdosing of active ingredient.

Formulations may thus be stored under normal storage conditions, and maintain their physical and/or chemical integrity. The phrase ‘maintaining physical and chemical integrity’ essentially means chemical stability and physical stability.

By ‘chemical stability’, we include that any formulation of the invention may be stored (with or without appropriate pharmaceutical packaging), under normal storage conditions, with an insignificant degree of chemical degradation or decomposition.

By ‘physical stability’, we include that the any formulation of the invention may be stored (with or without appropriate pharmaceutical packaging), under normal storage conditions, with an insignificant degree of physical transformation, such as sedimentation as described above, or changes in the nature and/or integrity of the coated particles, for example in the coating itself or the active ingredient (including dissolution, solvatisation, solid state phase transition, etc.).

Examples of ‘normal storage conditions’ for formulations of the invention include temperatures of between about −50° C. and about +80° C. (preferably between about −25° C. and about +75° C., such as about 50° C.), and/or pressures of between about 0.1 and about 2 bars (preferably atmospheric pressure), and/or exposure to about 460 lux of UV/visible light, and/or relative humidities of between about 5 and about 95% (preferably about 10 to about 40%), for prolonged periods (i.e. greater than or equal to about twelve, such as about six months).

Under such conditions, formulations of the invention may be found to be less than about 15%, more preferably less than about 10%, and especially less than about 5%, chemically and/or physically degraded/decomposed, as appropriate. The skilled person will appreciate that the above-mentioned upper and lower limits for temperature and pressure represent extremes of normal storage conditions, and that certain combinations of these extremes will not be experienced during normal storage (e.g. a temperature of 50° C. and a pressure of 0.1 bar).

Formulations of the invention may comprise between about 1% to about 99%, such as between about 10% (such as about 20%, e.g. about 50%) to about 90% by weight of the coated particles with the remainder made up by carrier system and/or other pharmaceutically-acceptable excipients.

Formulations of the invention may be in the form of a liquid, a sol or a gel, which is administrable via a surgical administration apparatus, e.g. a needle, a catheter or the like, to form a depot formulation.

In any event, the preparation of suitable formulations may be achieved non-inventively by the skilled person using routine techniques. Formulations of the invention and dosage forms comprising them, may thus be formulated with conventional pharmaceutical additives and/or excipients used in the art for the preparation of pharmaceutical formulations, and thereafter incorporated into various kinds of pharmaceutical preparations and/or dosage forms using standard techniques (see, for example, Lachman et al., ‘The Theory and Practice of Industrial Pharmacy’, Lea & Febiger, 3^{rd} edition (1986); ‘Remington: The Science and Practice of Pharmacy’, Troy (ed.), University of the Sciences in Philadelphia, 21^{st} edition (2006); and/or ‘Aulton's Pharmaceutics: The Design and Manufacture of Medicines’, Aulton and Taylor (eds.), Elsevier, 4^{th} edition, 2013), and the documents referred to therein, the relevant disclosures in all of which documents are hereby incorporated by reference.

According to a further aspect of the invention there is provided a process for the preparation of a formulation of the invention which comprises mixing together the coated particles as described herein with the aqueous carrier system, for example as described herein.

For parenteral administration, such as subcutaneous and/or intramuscular injections, the formulations of the invention may be presented in the form of sterile injectable and/or infusible dosage forms administrable via a surgical administration apparatus (e.g. a syringe with a needle for injection, a catheter or the like), to form a depot formulation.

There is further provided an injectable and/or infusible dosage form comprising a formulation of the invention, wherein said formulation is contained within a reservoir that is connected to, and/or is associated with, an injection or infusion means (e.g. a syringe with a needle for injection, a catheter or the like).

Alternatively, formulations of the invention can be stored prior to being loaded into a suitable injectable and/or infusible dosing means (e.g. a syringe with a needle for injection), or may even be prepared immediately prior to loading into such a dosing means.

Sterile injectable and/or infusible dosage forms may thus comprise a receptacle or a reservoir in communication with an injection or infusion means into which a formulation of the invention may be pre-loaded, or may be loaded at a point prior to use, or may comprise one or more reservoirs, within which coated particles of the formulation of the invention and the aqueous carrier system are housed separately, and in which admixing occurs prior to and/or during injection or infusion.

There is thus further provided a kit of parts comprising:
(a) coated particles of the formulation of the invention; and
(b) a carrier system of the formulation of the invention,
   as well as a kit of parts comprising coated particles of the formulation of the invention along with instructions to the end user to admix those particles with a carrier system according to the invention.

There is further provided a pre-loaded injectable and/or infusible dosage form as described herein above, but modified by comprising at least two chambers, within one of which chamber is located the coated particles of the formulation of the invention and within the other of which is located the aqueous carrier system of the formulation of the invention, wherein admixing, giving rise to a suspension or otherwise, occurs prior to and/or during injection or infusion.

Formulations of the invention may be used in human medicine. Formulations of the invention are particularly useful in any indication in which a biologic active drug is either approved for use in, or otherwise known to be useful in.

Formulations of the invention are indicated in the therapeutic, palliative, and/or diagnostic treatment, as well as the prophylactic treatment (by which we include preventing and/or abrogating deterioration and/or worsening of a condition) of any conditions for which the biologic active agent is known to treat.

Injection of formulations of the invention may cause a mild inflammatory response. Such a response may be alleviated by co-administration with an antiinflammatory agent that is suitable for injection.

Appropriate antiinflammatory agents that may be employed in this regard include butylpyrazolidines (such as phenylbutazone, mofebutazone, oxyphenbutazone, clofezone, kebuzone and suxibuzone); acetic acid derivatives and related substances (indomethacin, sulindac, tolmetin, zomepirac, diclofenac, alclofenac, bumadizone, etodolac, lonazolac, fentiazac, acemetacin, difenpiramide, oxametacin, proglumetacin, ketorolac, aceclofenac and bufexamac); oxicams (such as piroxicam, tenoxicam, droxicam, lornoxicam and meloxicam); propionic acid derivatives (such as ibuprofen, naproxen, ketoprofen, fenoprofen, fenbufen, benoxaprofen, suprofen, pirprofen, flurbiprofen, indoprofen, tiaprofenic acid, oxaprozin, ibuproxam, dexibuprofen, flunoxaprofen, alminoprofen, dexketoprofen, vedaprofen, carprofen and tepoxalin); fenamates (such as mefenamic acid, tolfenamic acid, flufenamic acid, meclofenamic acid and flunixin), coxibs (such as celecoxib, rofecoxib, valdecoxib, parecoxib, etoricoxib, lumiracoxib, firocoxib, robenacoxib, mavacoxib and cimicoxib); other non-steroidal antiinflammatory agents (such as nabumetone, niflumic acid, azapropazone, glucosamine, benzydamine, glucosaminoglycan polysulfate, proquazone, orgotein, nimesulide, feprazone, diacerein, morniflumate, tenidap, oxaceprol, chondroitin sulfate, pentosan polysulfate and aminopropionitrile); corticosteroids (such as 11-dehydrocorticosterone, 11-deoxycorticosterone, 11-deoxycortisol, 11-ketoprogesterone, 11β-hydroxypregnenolone, 11β-hydroxyprogesterone, 11β, 170,21-trihydroxypregnenolone, 17a,21-dihydroxypregnenolone, 17α-hydroxypregnenolone, 17α-hydroxyprogesterone, 18-hydroxy-11-deoxycorticosterone, 18-hydroxycorticosterone, 18-hydroxyprogesterone, 21-deoxycortisol, 21-deoxycortisone, 21-hydroxypregnenolone (prebediolone), aldosterone, corticosterone (17-deoxycortisol), cortisol (hydrocortisone), cortisone, pregnenolone, progesterone, flugestone (flurogestone), fluorometholone, medrysone (hydroxymethylprogesterone), prebediolone acetate (21-acetoxypregnenolone), chloroprednisone, cloprednol, difluprednate, fludrocortisone, fluocinolone, fluperolone, fluprednisolone, loteprednol, methylprednisolone, prednicarbate, prednisolone, prednisone, tixocortol, triamcinolone, alclometasone, beclometasone, betamethasone, clobetasol, clobetasone, clocortolone, desoximetasone, dexamethasone, diflorasone, difluocortolone, fluclorolone, flumetasone, fluocortin, fluocortolone, fluprednidene, fluticasone, fluticasone furoate, halometasone, meprednisone, mometasone, mometasone furoate, paramethasone, prednylidene, rimexolone, ulobetasol (halobetasol), amcinonide, budesonide, ciclesonide, deflazacort, desonide, formocortal fluclorolone acetonide (flucloronide), fludroxycortide (flurandrenolone, flurandrenolide), flunisolide, fluocinolone acetonide, fluocinonide, halcinonide and triamcinolone acetonide); quinolines (such as oxycinchophen); gold preparations (such as sodium aurothiomalate, sodium aurothiosulfate, auranofin, aurothioglucose and aurotioprol); penicillamine and similar agents (such as bucillamine); and antihistamines (such as akrivastin, alimemazin, antazolin, astemizol, azatadin, azelastin, bamipin, bilastin, bromdifenhydramin, bromfeniramin, buklizin, cetirizin, cinnarizine, cyklizin, cyproheptadine, deptropine, desloratadin, dexbromfeniramin, dexklorfeniramin, difenylpyralin, dimenhydrinat, dimetinden, doxylamin, ebastin, epinastin, fenindamin, feniramin, fexofenadin, histapyrrodin, hydroxietylprometazin, isotipendyl, karbinoxamin, ketotifen, kifenadin, klemastin, klorcyklizin, klorfenamin, klorfenoxamin, kloropyramin, levocetirizin, loratadin, mebhydrolin, mekitazin, meklozin, mepyramin, metapyrilen, metdilazin, mizolastin, oxatomide, oxomemazine, pimetixen, prometazin, pyrrobutamin, rupatadin, sekifenadin, talastin, tenalidin, terfenadin, tiazinam, tietylperazin, tonzylamin, trimetobenzamid, tripelennamin, triprolidine and tritokvalin). Combinations of any one or more of the above-mentioned antiinflammatory agents may be used.

Preferred antiinflammatory agents include non-steroidal anti-inflammatory drugs, such as diclofenac, ketoprofen, meloxicam, aceclofenac, flurbiprofen, parecoxib, ketoralac tromethamine or indomethacin.

Subjects may receive (or may already be receiving) one or more of the aforementioned antiinflammatory agents, separate to a formulation of the invention, by which we mean receiving a prescribed dose of one or more of those antiinflammatory agents, prior to, in addition to, and/or following, treatment with a formulation of the invention.

When biologic active agents are ‘combined’ with such antiinflammatory agents, the active ingredients may be administered together in the same formulation, or administered separately (simultaneously or sequentially) in different formulations (hereinafter referred to as ‘combination products’).

Such combination products provide for the administration of biologic active agent in conjunction with the antiinflammatory agent, and may thus be presented either as separate formulations, wherein at least one of those formulations is a formulation of the invention, and at least one comprises the antiinflammatory agent in a separate formulation, or may be presented (i.e. formulated) as a combined preparation (i.e. presented as a single formulation including biologic active agent and the antiinflammatory agent).

In this respect an antiinflammatory agent may be co-presented with biologic active agent at an appropriate dose in one or more of the cores that form part of a formulation of the invention as hereinbefore described, or may be formulated using the same or a similar process for coating to that described hereinbefore for biologic active agent, which may allow for the release of the antiinflammatory agent over the same, or over a different timescale.

Thus, there is further provided a pharmaceutical formulation of the invention that further comprises an antiinflammatory agent.

In such formulations of the invention, the antiinflammatory agent may be included by:
(1) formulating along with the biologic active agent within the solid cores of a formulation of the invention (which formulation is hereinafter referred to as a ‘combined core preparation’); or
(2) dissolving it, and/or suspending it, within the aqueous carrier system of a formulation of the invention (which formulation is hereinafter referred to as a ‘combination preparation’).

In embodiment (2) above, the antiinflammatory agent may be presented in a formulation of the invention in any form in which it is separate to the biologic active agent-containing cores. This may be achieved by, for example, dissolving or suspending that active ingredient directly in the aqueous medium of a formulation of the invention, or by presenting it in a form in which its release can, like the biologic active agent, also be controlled following injection.

The latter option may be achieved by, for example, providing the antiinflammatory agent in the form of additional particles suspended in the aqueous carrier system of formulation of the invention, which additional particles have a weight-, number-, or volume-based mean diameter that is between amount 10 nm and about 700 μm, and comprise cores comprising the antiinflammatory agent, which cores are coated, at least in part, by one or more coating materials as hereinbefore described (which formulation is hereinafter referred to as a ‘combination suspension’).

There is further provided a pharmaceutical formulation of the invention that is in the form of a kit of parts comprising components:
(A) a pharmaceutical formulation of the invention; and
(B) a pharmaceutical formulation, comprising an antiinflammatory agent, which Components (A) and (B) are each provided in a form that is suitable for administration in conjunction with the other.

Although Component (B) of a kit of parts as presented above may be different in terms its chemical composition and/or physical form from Component (A) (i.e. a formulation of the invention), it may also be in a form that is essentially the same or at least similar to a biologic active agent-containing formulation of the invention, that is in the form of a plurality of particles suspended in an (e.g. aqueous) carrier system, which particles:
(a) have a weight-, number-, or volume-based mean diameter that is between amount 10 nm and about 700 μm; and
(b) comprise solid cores comprising that antiinflammatory agent, which cores are coated, at least in part, by one or more coatings of (e.g. inorganic) material.

In addition, although, in such preferred kits of parts, and the combination suspensions presented under embodiment (2) above, the coated cores comprising the antiinflammatory agent may be different in terms of their chemical composition(s) and/or physical form(s), it is preferred that the coating of inorganic material that is employed is the same or similar to that employed in biologic active agent-containing formulations of the invention, which means that the antiinflammatory agent is coated by one or more inorganic coatings as hereinbefore described, for example one or more inorganic coating materials comprising one or more metal-containing, or metalloid-containing, compounds, such as a metal, or metalloid, oxide, for example iron oxide, titanium dioxide, zinc sulphide, more preferably zinc oxide, silicon dioxide and/or aluminium oxide, which coating materials may (on an individual or a collective basis) consist essentially (e.g. are greater than about 80%, such as greater than about, 90%, e.g. about 95%, such as about 98%) of such oxides, and more particularly inorganic coatings comprising a mixture of:
(i) zinc oxide; and
(ii) one or more other metal and/or metalloid oxides,
   wherein the atomic ratio ((i):(ii)) is at least about 1:6 and up to and including about 6:1.

Preferably, the atomic ratio ((i):(ii)) is at least about 1:1 and up to and including about 6:1.

In any event, and for the avoidance of doubt, all aspects, including preferred aspects, disclosed and/or claimed herein for in biologic active agent-containing formulations of the invention are equally applicable as aspects and/or preferences for coated cores comprising one or more of the antiinflammatory agents described above. For the avoidance of doubt, such aspects, preferences and features, alone or in combination, are hereby incorporated by reference to these aspects of the invention.

All combination products, including combined core preparations, combination suspensions and kits of parts described above may thus be used in human medicine and, in particular, any indication in which the biologic active agent is either approved for use in, or otherwise known to be useful in.

According to a further aspect of the invention, there is provided a method of making a kit of parts as defined above, which method comprises bringing Component (A), as defined above, into association with a Component (B), as defined above, thus rendering the two components suitable for administration in conjunction with each other.

By bringing the two components ‘into association with’ each other, we include that Components (A) and (B) of the kit of parts may be:
(i) provided as separate formulations (i.e. independently of one another), which are subsequently brought together for use in conjunction with each other in combination treatment; or
(ii) packaged and presented together as separate components of a ‘combination pack’ for use in conjunction with each other in combination treatment.

Thus, there is further provided a kit of parts as hereinbefore defined in which Components (A) and (B) are packaged and presented together as separate components of a combination pack, for use in conjunction with each other in combination treatment, as well as a kit of parts comprising:
(I) one of Components (A) and (B) as defined herein; together with
(II) instructions to use that component in conjunction with the other of the two components.

As alluded to above, the kits of parts described herein may comprise more than one formulation including an appropriate quantity/dose of biologic active agent and/or more than one formulation including an appropriate quantity/dose of the antiinflammatory agent, in order to provide for repeat dosing as hereinbefore described.

In this respect, with respect to the kits of parts as described herein, by ‘administration in conjunction with’, we include that Components (A) and (B) of the kit are administered, sequentially, separately and/or simultaneously, over the course of treatment of the condition.

Thus, the term ‘in conjunction with’ includes that one or other of the two formulations may be administered (optionally repeatedly) prior to, after, and/or at the same time as, administration of the other component. When used in this context, the terms ‘administered simultaneously’ and ‘administered at the same time as’ include that individual doses of biologic active agent and antiinflammatory agent are administered within 48 hours (e.g. 24 hours) of each other.

In respect of any of the above combination products according to the invention, the respective formulations are administered (or, in the case of the kit of parts, the two components are administered, optionally repeatedly, in conjunction with each other) in a manner that may enable a beneficial effect for the subject, that is greater, over the course of the treatment of the condition, than if a formulation (e.g. a formulation of the invention) comprising biologic active agent alone is administered (e.g. repeatedly, as described herein) in the absence of the other component, over the same course of treatment.

Determination of whether a combination product provides a greater beneficial effect in respect of, and over the course of treatment will depend upon the condition to be treated and/or its severity, but may be achieved routinely by the skilled person.

The physician may then administer one or more of:
Component (B) of a kit of parts as described above,
a combined core preparation,
a combination preparation, and/or
a combination suspension
   as described above, any of which comprises an antiinflammatory agent as hereinbefore described.

The amount of the antiinflammatory agent that may be employed in combination products according to the invention must be sufficient so exert its pharmacological effect.

Doses of such antiinflammatory ingredients that may be administered to a patient should thus be sufficient to affect a therapeutic response over a reasonable and/or relevant timeframe. One skilled in the art will recognize that the selection of the exact dose and composition and the most appropriate delivery regimen will also be influenced by not only the nature of the antiinflammatory ingredient, but also inter alia the pharmacological properties of the formulation, the route of administration, the nature and severity of the condition being treated, and the physical condition and mental acuity of the recipient, as well as the age, condition, body weight, sex and response of the patient to be treated, and the stage/severity of the disease, as well as genetic differences between patients.

As administration of formulations of the invention may be continuous or intermittent (e.g. by bolus injection), dosages of such antiinflammatory ingredients may also be determined by the timing and frequency of administration.

In any event, the medical practitioner, or other skilled person, will be able to determine routinely the actual dosage of any particular additional active ingredient, which will be most suitable for an individual patient, and doses of the relevant additional active ingredients mentioned above include those that are known in the art and described for the drugs in question to in the medical literature, such as Martindale—The Complete Drug Reference, 38^{th} Edition, Pharmaceutical Press, London (2014) and the documents referred to therein, the relevant disclosures in all of which documents are hereby incorporated by reference.

The use of formulations of the invention may control the dissolution rate of the biologic active drug and affect the pharmacokinetic profile by reducing any burst effect as hereinbefore defined (e.g. a concentration maximum shortly after administration), and/or by reducing Cₘₐₓ in a plasma concentration-time profile.

Formulations of the invention may also provide a release and/or pharmacokinetic profile that increases the length of release of biologic active drug from the formulation.

These factors not only reduce the frequency at or over which the formulation needs to be administered to a subject, but also allows the subject more time as an out-patient, and so to have a better quality of life.

The formulations of the invention also has the advantage that by controlling the release of active ingredient at a steady rate over a prolonged period of time, a lower daily exposure to e.g., a cytotoxic drug is provided, which is expected to reduce unwanted side effects.

The formulations and processes described herein may also have the advantage that, in the treatment of the relevant conditions, they may be more convenient for the physician and/or patient than, be more efficacious than, be less toxic than, have a broader range of activity than, be more potent than, produce fewer side effects than, or that it may have other useful pharmacological properties over, any similar treatments known in the prior art.

Wherever the word ‘about’ is employed herein, for example in the context of amounts (e.g. numbers, concentrations, dimensions (sizes and/or weights), doses, time periods, pharmacokinetic parameters, etc.), relative amounts (percentages, weight ratios, size ratios, atomic ratios, aspect ratios, proportions, factors or fractions, etc.), relative humidities, lux, temperatures or pressures, it will be appreciated that such variables are approximate and as such may vary by ±15%, such as ±10%, for example ±5% and preferably ±2% (e.g. ±1%) from the numbers specified herein. This is the case even if such numbers are presented as percentages in the first place (for example ‘about 15%’ may mean ±15% about the number 10, which is anything between 8.5% and 11.5%).

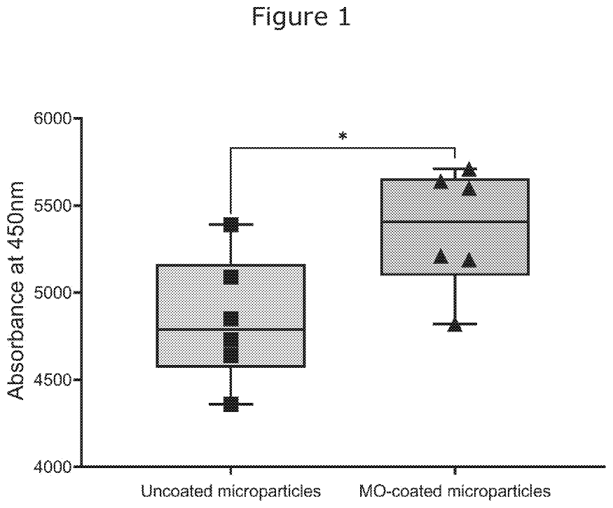

The invention is illustrated, but in no way limited, by the following examples with reference to the figures, in which FIG. 1 shows the absorbance measured at 450 nm in UV-VIS plate reader after ELISA on coated and uncoated spray-dried microparticles comprising the monoclonal antibody ATH3G10.

### EXAMPLES

### Example 1

### Mixed Oxide Coated Anakinra Microparticles I.

Samples of microparticles of anakinra will be prepared by spray-drying together with trehalose to obtain mean particle diameters of 5 μm as determined by laser diffraction.

The powder will be loaded to an ALD reactor (Picosun, SUNALE™ R-series, Espoo, Finland) where 24 ALD cycles will be performed at a reactor temperature of 50° C. The coating sequence will be three ALD cycles employing diethyl zinc and water as precursors for three ALD cycles, followed by one cycle of trimethylaluminium and water, repeated six times, to forming a mixed oxide layer of with an atomic ratio of zinc:aluminium of 3:1. The first layer is expected to be about 5 nm in thickness (as estimated from the number of ALD cycles).

The powder will be removed from the reactor and deagglomerated by means of forcing the powder through a polymeric sieve with a 20 μm mesh size using a sonic sifter.

The resultant deagglomerated powder will be re-loaded into the ALD reactor and further 24 ALD cycles will be performed as before forming a second layer of mixed oxide at the aforementioned ratio, followed by extraction from the reactor and deagglomeration by means of sonic sifting as above, followed by reloading to form a third layer, deagglomeration and then reloading to form a final, fourth layer.

To determine the drug load (i.e. w/w % of anakinra in the powder), it is planned to determine the drug load using methods known in the art. The nanoshell coatings will be dissolved in 2 M phosphoric acid in DMSO and the slurry then diluted with DMSO, before filtration (0.2 μm RC, Lab Logistics Group, Germany) and further analyzed with HPLC (n=2).

### Example 2

### Mixed Oxide Coated Anakinra Microparticles II

The same procedure as described in Example 1 will be conducted to produce microparticles coated with mixed oxide coating comprising an atomic ratio of zinc:aluminium of 2:1.

The coating sequence will be two ALD cycles employing diethyl zinc and water as precursors, followed by one cycle of trimethylaluminium and water, repeated ten times removal of the coated powder from the reactor, deagglomeration, reloading and repeating the same coating sequence, removal, deagglomeration until 4 sets of 30 cycles in total has been provided.

### Example 3

### Aluminium Oxide Coated Anakinra Microparticles

The same microparticles that will be coated with the mixed oxide coating as described in Example 1 will be coated with a pure aluminium oxide coating. 30 ALD cycles will be performed prior to removing the coated powder from the reactor and deagglomerated as described in Example 1. The resultant deagglomerated powder will be reloaded into the ALD reactor and subjected to a further 30 ALD cycles, followed by extraction deagglomeration, reloading and repeating the same coating sequence, removal, deagglomeration until 4 sets of 30 cycles in total have been provided.

### Example 4

### Zinc Oxide Coated Anakinra Microparticles

The same microparticles that will be coated with the mixed oxide coating as described in Example 1 will be coated with a pure zinc oxide coating. 30 ALD cycles will be performed prior to removing the coated powder from the reactor and deagglomerated as described in Example 1. The resultant deagglomerated powder will be reloaded into the ALD reactor and subjected to a further 30 ALD cycles, followed by extraction deagglomeration, reloading and repeating the same coating sequence, removal, deagglomeration until 4 sets of 30 cycles in total have been provided.

### Example 5

### Formulations of the Invention I

The same microparticles that will be coated with the oxide coatings as described in Example 1, Example 2, Example 3 and Example 4 will be suspended in a commercially-available aqueous vehicle, Hyonate® vet (Boehringer Ingelheim Animal Health, France), which is a veterinary medicinal product used in injecting animals, such as horses, comprising a sterile, isotonic, phosphate buffered solution of 10 mg/ml of sodium hyaluronate (pH 7.4).

The concentration of anakinra in the formulation is 10 mg/mL, which accords to 10 mg/kg body weight of a Sprague-Dawley rat.

### Example 6

### Formulation of the Invention II

A suspension of coated microparticles of anakinra (prepared according to the process described in Example 4 above) will be suspended in an aqueous vehicle comprising 0.1% (w/w) of Polysorbate 20, 0.25% (w/w), sodium carboxylmethyl cellulose in a phosphate buffered saline solution (pH 7.4).

The concentration of anakinra in the formulation is 10 mg/mL, which accords to 10 mg/kg body weight of a Sprague-Dawley rat.

### Example 7

### Formulations of the Invention III

A suspension of uncoated microparticles of anakinra same sort of uncoated microparticles that are used for the coated microparticles in Examples 1-4) will be suspended in a commercially-available aqueous vehicle, Hyonate® vet to a concentration of azacitidine in the formulation of 10 mg/mL, which accords to 10 mg/kg body weight of a Sprague-Dawley rat.

### Example 8

### In Vivo Rat Study I

Thirty-six male Sprague Dawley rats will be supplied e.g. by Charles River (UK). The animals will be divided randomly into six animals per group.

The intended administration area will be clipped free from hair prior to injection and the injection site will be marked. As set out in Table 1 below, the suspensions described in Example 5 (Group 1 suspension of particles according to Example 1, Group 2 suspension of particles according to Example 2, Group 3 suspension of particles according to Example 3 and Group 4 suspension of particles according to Example 4), Example 6 (Group 5) and Example 7 (Group 6) will be drawn into a 1 mL BD syringe and single, subcutaneous injections (ca. 0.3 mL) will be administered through a 23G needle (BD microlance) into the flank of each rat. Administration will be performed no more than 30 minutes after preparation of the formulations.

TABLE 1

| Group | Description | Dose |
|---|---|---|
| 1 | Mixed (3:1) coated particles in Hyonate (Ex. 1 and 5) | 10 |
| 2 | Mixed (2:1) coated particles in Hyonate (Ex. 2 and 5) | 10 |
| 3 | Aluminium oxide coated particles in Hyonate | 10 |
| | (Ex. 3 and 5) | |
| 4 | Zinc oxide coated particles in Hyonate (Ex. 4 and 5) | 10 |
| 5 | Zinc oxide coated particles in Polysorbate 20 and sodium | 10 |
| | carboxylmethyl cellulose (Ex. 6) | |
| 6 | Uncoated particles in Hyonate | 10 |

Blood samples (ca 0.2 mL) will be collected from the tail vein into K₂EDTA (dipotassium ethylenediaminetetraacetic acid) tubes at the following time-points: 0.5, 1, 3, 6, 12, 24, 48, 72, 120, 168, 240, and 336 h post-dose. Actual sampling times will be recorded. As soon as practically possible following blood sampling, plasma will be separated by centrifugation (1500 g for 10 min at 4° C.), which will be stored at −80° C. until analysis is conducted.

Following study completion, all plasma samples will be shipped for analysis having been deep frozen on dry ice. Animals will be sacrificed on the last day of the study.

Plasma concentration of anakinra will be determined using HPLC-MS/MS.

It is expected that relative bioavailabilities of the formulations are comparable with the uncoated biologic active drug.

Pharmacokinetic analysis of ankinra in plasma will be performed according to standard non-compartmental approach using Microsoft Excel for Mac (16.43, Microsoft, Redmond, Washington, USA). Maximum concentration, Cₘₐₓ, and related time, tₘₐₓ, will be the coordinates of the highest concentration of the time course. tₗₐₛₜ will be the time of the last detectable concentrations. The area under concentration vs. time curve up to the last detectable concentration, AUCₗₐₛₜ, will be calculated using the linear trapezoidal rule.

### Results

Dose-normalised plasma concentrations of anakinra after single subcutaneous administration of the various formulations will be presented. The plasma pharmacokinetic parameters will also be presented as mean values for the group of 6 rats (with standard deviations provided in parentheses).
dose is expressed in mg/kg body weight of the rat
‘tₘₐₓ’ is the time to peak concentration expressed in hours
‘Cₘₐₓ’ is the maximum concentration found in analysis expressed in μg/mL
‘tₗₐₛₜ’ is the time of the last detectable concentration expressed in hours
‘t_{1/2,z}’ is the terminal half-life expressed in hours
‘AUC_{∞}’ is the area under concentration vs. time curve up to infinite time expressed in μg*h/mL
‘F’ is the relative bioavailability expressed as a percentage
‘Cₘₐₓ/D’ is the maximum concentration normalized to 1 mg/kg expressed in μg/mL/mg/kg body weight of the rat
‘AUCₗₐₛₜ/D’ is the area under blood concentration vs. time curve up to the last detectable concentration normalized to 1 mg/kg expressed in μg*h/mL/mg/kg body weight of the rat
‘AUC_{∞}/D’ is the area under concentration vs. time curve up to infinite time normalized to 1 mg/kg expressed in μg*h/mL/mg/kg body weight of the rat
‘Fr. Rel.₀₋₁₂n’ is the fraction released during the first twelve hours of the area under concentration vs. time curve up to infinite time expressed as a percentage.

The following order would be suspected in terms of drug release (fastest to slowest):
Group 6
Group 4
Group 1
Group 2
Group 3

The formulation for Group 5, i.e. the formulation of Example 6 is likely to form a gel which is expected to be difficult to administer.

### Example 9

### Formulations of the Invention IV

The same microparticles that will be coated with the oxide coatings as described in Example 1, Example 2, Example 3 and Example 4 will be suspended in a commercially-available aqueous vehicle, Hyonate® vet (Boehringer Ingelheim Animal Health, France), which is a veterinary medicinal product used in injecting animals, such as horses, comprising a sterile, isotonic, phosphate buffered solution of 200 mg/ml of sodium hyaluronate (pH 7.4).

The concentration of anakinra in the formulation is 200 mg/mL, which accords to 200 mg/kg body weight of a Sprague-Dawley rat.

### Example 10

### Formulation of the Invention V

A suspension of coated microparticles of anakinra (prepared according to the process described in Example 4 above) will be suspended in an aqueous vehicle comprising 0.1% (w/w) of Polysorbate 20, 0.25% (w/w), sodium carboxylmethyl cellulose in a phosphate buffered saline solution (pH 7.4).

The concentration of anakinra in the formulation is 200 mg/ml, which accords to 200 mg/kg body weight of a Sprague-Dawley rat.

### Example 11

### Formulations of the Invention VI

A suspension of uncoated microparticles of anakinra same sort of uncoated microparticles that are used for the coated microparticles in Examples 1-4) will be suspended in a commercially-available aqueous vehicle, Hyonate® vet to a concentration of azacitidine in the formulation of 10 mg/ml, which accords to 200 mg/kg body weight of a Sprague-Dawley rat.

### Example 12

### In Vivo Rat Study II

Thirty-six male Sprague Dawley rats will be supplied e.g. by Charles River (UK). The animals will be divided randomly into six animals per group.

The intended administration area will be clipped free from hair prior to injection and the injection site will be marked. As set out in Table 2 below, the suspensions described in Example 9 (Group 1 suspension of particles according to Example 1, Group 2 suspension of particles according to Example 2, Group 3 suspension of particles according to Example 3 and Group 4 suspension of particles according to Example 4), Example 10 (Group 5) and Example 11 (Group 6) will be drawn into a 1 mL BD syringe and single, subcutaneous injections (ca. 0.3 mL) will be administered through a 23G needle (BD microlance) into the flank of each rat. Administration will be performed no more than 30 minutes after preparation of the formulations.

TABLE 2

| Group | Description | Dose |
|---|---|---|
| 1 | Mixed (3:1) coated particles in Hyonate (Ex. 1 and 5) | 200 |
| 2 | Mixed (2:1) coated particles in Hyonate (Ex. 2 and 5) | 200 |
| 3 | Aluminium oxide coated particles in Hyonate | 200 |
| | (Ex. 3 and 5) | |
| 4 | Zinc oxide coated particles in Hyonate (Ex. 4 and 5) | 200 |
| 5 | Zinc oxide coated particles in Polysorbate 20 and sodium | 200 |
| | carboxylmethyl cellulose (Ex. 6) | |
| 6 | Uncoated particles in Hyonate | 200 |

Blood samples (ca 0.2 mL) will be collected from the tail vein into K₂EDTA (dipotassium ethylenediaminetetraacetic acid) tubes at the following time-points: 0.5, 1, 3, 6, 12, 24, 48, 72, 120, 168, 240, and 336 h post-dose. Actual sampling times will be recorded. As soon as practically possible following blood sampling, plasma will be separated by centrifugation (1500 g for 10 min at 4° C.), which will be stored at −80° C. until analysis is conducted.

Following study completion, all plasma samples will be shipped for analysis having been deep frozen on dry ice. Animals will be sacrificed on the last day of the study.

Plasma concentration of anakinra will be determined using HPLC-MS/MS.

It is expected that relative bioavailabilities of the formulations are comparable with the uncoated biologic active drug.

Pharmacokinetic analysis of ankinra in plasma will be performed according to standard non-compartmental approach using Microsoft Excel for Mac (16.43, Microsoft, Redmond, Washington, USA). Maximum concentration, Cₘₐₓ, and related time, tₘₐₓ, will be the coordinates of the highest concentration of the time course. tₗₐₛₜ will be the time of the last detectable concentrations. The area under concentration vs. time curve up to the last detectable concentration, AUCₗₐₛₜ, will be calculated using the linear trapezoidal rule.

### Results

Dose-normalised plasma concentrations of anakinra after single subcutaneous administration of the various formulations will be presented. The plasma pharmacokinetic parameters will also be presented as mean values for the group of 6 rats (with standard deviations provided in parentheses).
dose is expressed in mg/kg body weight of the rat
‘tₘₐₓ’ is the time to peak concentration expressed in hours
‘Cₘₐₓ’ is the maximum concentration found in analysis expressed in μg/mL
‘Last’ is the time of the last detectable concentration expressed in hours
‘t_{1/2,z}’ is the terminal half-life expressed in hours
‘AUC_{∞}’ is the area under concentration vs. time curve up to infinite time expressed in μg*h/mL
‘F’ is the relative bioavailability expressed as a percentage
‘Cₘₐₓ/D’ is the maximum concentration normalized to 1 mg/kg expressed in μg/mL/mg/kg body weight of the rat
‘AUCₗₐₛₜ/D’ is the area under blood concentration vs. time curve up to the last detectable concentration normalized to 1 mg/kg expressed in μg*h/mL/mg/kg body weight of the rat
‘AUC_{∞}/D’ is the area under concentration vs. time curve up to infinite time normalized to 1 mg/kg expressed in μg*h/mL/mg/kg body weight of the rat
‘Fr. Rel.₀₋₁₂n’ is the fraction released during the first twelve hours of the area under concentration vs. time curve up to infinite time expressed as a percentage.

The following order would be suspected in terms of drug release (fastest to slowest):
Group 6
Group 4
Group 1
Group 2
Group 3

The formulation for Group 5, i.e. the formulation of Example 10 is likely to form a gel which is expected to be difficult to administer.

### Example 13

### Coated Monoclonal Antibodies

ATH3G10 clinical trial material, a solution of fully human IgG1 monoclonal antibody with specific affinity to phosphocholine (PC mAb), dissolved at 20 mg/ml in a solution of 125 mM sodium chloride, 100 mM glycine, and 25 mM sodium acetate in water, pH 5.5 was obtained from Athera Biotechnologies AB (Stockholm, Sweden). Generation of this specific monoclonal antibody is described in de Vries et al. (2021), J. Intern. Med. 290(1), pp. 141-156, and general procedures to generate anti-phosphorylcholine antibodies are described in the art, e.g., U.S. Pat. No. 5,455,032. In summary, the described antibody can be generated by synthesizing DNA encoding the antibody sequence and cloning into a plasmid for transfection into 293T cells. The cells will then transiently generate antibodies which can be purified using e.g., a protein-A Sepharose column.

For large scale production, a stable cell line has been developed. Gene-optimised DNA sequences encoding the signal peptides and coding regions of the variable regions of the heavy chain (VH) and light chain (VL) of the X19-A05 antibody were generated by Geneart A G (Regensburg, Germany). The DNA sequence encoding the VH was cloned into a vector containing gene-optimised cDNA sequence encoding the human IgGiza constant region except for the codon encoding the C-terminal lysine residue. DNA sequences encoding the VL were cloned into a vector containing gene-optimised cDNA sequence encoding the human kappa constant region. CHOK1SV host cells were transfected using a single vector encoding both complete heavy and light chain genes to generate stable GS-CHO transfectant pools expressing the PC mAb. Cell line 3G10 was selected for cGMP manufacture of the PC-mAb antibody designated ATH3G10. Vector construction, cell-line generation, and antibody production were performed by Lonza Biologics plc (Slough, UK).

The ATH3G10 clinical trial material was reformulated by dialysis through 20 kDa MWCO membranes. The dialysis process was conducted against a 1:10 volume of water for injection for three hours, after which the dialysate was replaced by a 1:10 volume of buffer comprising 5 mg/mL DL-histidine in water, adjusted to pH 6.0 using glacial acetic acid. After three hours, the dialysate was replaced by a 1:10 volume of buffer comprising 3.3 mg/mL trehalose and 4.5 mg/mL DL-histidine in water, adjusted to pH 6.0 using glacial acetic acid, and the dialysis continued for another 12 hours. The dialysis process was followed by measuring the chloride concentrations using titrator test strips (30-600 mg/L Cl⁻). Following dialysis, the composition of the retentate was 0.8-1.6% trehalose, 3.4-3.9% DL-histidine, 0.4-0.5% glycine, 0.3-0.5% sodium chloride, and 0.1% sodium acetate in water. Additional trehalose was added to obtain a final concentration of 3.7%-4.1% trehalose in the retentate. The retentate was then spray-dried using a Büchi B-290 Mini (Essen, Germany) with an inlet temperature of 110° C., aspirator rate of 100%, pump rate of 4.6 mL/min, spray gas (N₂) volume flow of 600-700 L/h, yielding an outlet temperature of 65-67° C. The spray-drying process took 45 minutes, and the yield was >90%. A fine white powder of amorphous particles with toroidal and conjoined toroidal morphologies was obtained, with mean particle diameters of 3-5 μm as determined by scanning electron microscopy.

Samples of microparticles of ATH3G10, a fully human IgG1 monoclonal antibody with specific affinity to phosphocholine (Athera Biotechnologies, Stockholm, Sweden), was prepared by spray-drying together with trehalose and DL-histidine to obtain particles with toroidal and conjoined toroidal morphologies, with mean particle diameters of 5 μm as determined by scanning electron microscopy.

The powder was loaded to an ALD reactor (Picosun, SUNALE™ R-series, Espoo, Finland) where 20 ALD cycles was performed at a reactor temperature of 30° C. The coating sequence was trimethyl aluminum and water as precursors which were pulsed into the reactor with the means of a stop-flow process. This means that each precursor was left in the reaction chamber without active pumping to be able to coat all the surfaces. The microparticles were subjected to 20 ALD cycles to produce a first layer of aluminium oxide.

The ALD reactor comprised a reaction chamber into which the microparticles were loaded. The ALD reactor further comprised precursor bottles containing each precursor separately, each precursor bottle being coupled to the reaction chamber via a valve. The ALD reactor also comprised a pump and associated piping for pumping an inert gas such as nitrogen through the reaction chamber, which pump was also coupled to the reaction chamber via a valve.

The ALD-cycle was performed as follows, wherein steps a to d represent a first cycle, subsequent cycles start from step a as specified in step e.
a. Reagent pulse 1:
   i. The valve on the piping between the pump and the ALD reactor was closed.
   ii. The valve on the water precursor bottle was opened for 0.5 s letting evaporated water fill the reaction chamber.
   iii. The valve to the water precursor bottle was closed and, before opening the pump valve again, the chamber was rested for 30 s (soaking time) to ensure the water vapor adsorbed onto the surface of the drug particles, presenting hydroxyl groups on the exterior or the particles.
   iv. The reactor was thereafter pumped for 9 s.
   v. Steps i-iv above were repeated 20 times.
b. Purging pulse: The chamber was purged with nitrogen in a continuous flow. Gaseous water, and organic gases were removed.
c. Reagent pulse 2:
   i. The valve on the piping between the pump and the ALD reactor was closed.
   ii. The valve on the trimethylaluminium precursor bottle was opened for 0.5 s letting evaporated metal containing precursor to fill the reaction chamber.
   iii. The valve to the precursor bottle was closed and, before opening to the pump again, the chamber was rested for 30 s (soaking time) to ensure the metal containing precursor vapor reacted with the hydroxyl groups on the surface of the drug particles.
   iv. The reactor was thereafter pumped for 9 s.
   V. Steps i-iv above was repeated 20 times.
d. Purging pulse: The chamber was purged with nitrogen in a continuous flow. Non-reacted reagents and organic gases were removed,
e. The cycle was repeated from step a-d 20 times.
   2. Next the powder was removed from the reactor and deagglomerated by means of sonic sifter (Tsutsui Sonic Agitated Sifting Machine SW-20AT) with a 20 μm mesh size sieve.
   3. The resultant deagglomerated powder was re-loaded into the ALD reactor and steps 1 were repeated once, forming a second layer of aluminium oxide.

### Analysis

The drug load (i.e. w/w % of ATH3G10 in the powder) was estimated from buffer replacement (via dialysis) through sodium chloride concentration as measured by chloride titrator strips. Combined with the estimated yield after spray-drying, the drug load in the uncoated particles was estimated to be approximately 69% ATH3G10, with the remainder comprising trehalose, histidine, water, glycine, sodium chloride, and sodium acetate (in descending order of fraction by mass). Drug load after coating was estimated gravimetrically by the powder mass before and after coating. The drug load of the coated microparticles was estimated to be approximately 44%.

### Example 14

### Coated Monoclonal Antibody Formulation

The microparticles from Example 13 above were suspended in a vehicle comprising 0.05% (w/v) polysorbate 20, 20 mM monopotassium phosphate, 0.8% (w/v) sodium chloride, 0.02% (w/v) potassium chloride, and water for injection, pH 7.2. The tested concentrations of ATH3G10 in the formulation were 132 and 200 mg/mL.

The formulation was tested with regards to injectability using a TA.XTplus Texture Analyser and Exponent software, which was used to measure the forces required to extend a piston.

In the method, the instrument piston was connected to a syringe piston (1 mL, 3-part luer lock, polypropylene housing) with a 25-gauge hypodermic needle (25 mm in length), filled with 1 mL of the formulation. For reference, the spray-dried (uncoated) microparticles were dissolved in a vehicle to the same ATH3G10 concentration and tested using this method. The vehicle for the reference solution comprised 125 mM sodium chloride, 100 mM glycine, 25 mM sodium acetate, and 10 mM phosphate in water, pH 5.8. During the test, the piston was extended at 10 mm/s until a threshold force of 50 N was measured, corresponding to full dispensing of the syringe content into air. A threshold of 15 N was considered as the maximum acceptable force required for injection.

### Results

The injectability test showed a higher required force for injection of the coated microparticle suspensions compared to the corresponding ATH3G10 solutions. However, the average force over the entire injection for suspensions of the coated microparticles corresponded to 132 mg/mL ATH3G10 through a 25-gauge hypodermic needle, which was acceptable. The average recorded force for this condition was 5.03±0.16 N. The corresponding average force recorded for the solution formulation at 132 mg/mL ATH3G10 was 3.97±0.04 N.

Thus, at a drug load of approximately 43% (which may be viewed as a worst-case scenario due to the high specific surface area of the particles generated by the spray-drying process), a highly concentrated dose can still be injected through a 25-gauge hypodermic needle.

These conditions are expected to be suitable for subcutaneous administration without the patient experiencing significant pain during the injection. Injectability of coated microparticle suspension formulation was not improved, but similar compared to an equivalent solution formulation. However, the mixed metal-oxide coating provides extended-release properties of the suspension formulation and could contribute to reductions in the required dosing frequency. Additionally, the mixed metal-oxide coating has been shown to act as an efficient moisture-barrier, which can prolong the shelf-life of the product.

### Example 15

### Affinity Test

To investigate whether the affinity of ATH3G10 towards its antigen is retained after being subjected to ALD coating, the coated ATH3G10 microparticles from Example 13 were tested using an indirect enzyme-linked immunosorbent assay (ELISA) and compared to uncoated ATH3G10.

Coated material (9 mg) was dispersed in 5 mL of ethylenediaminetetraacetic acid solution (0.02% in 0.5 mM Dulbecco's phosphate buffered saline). After overhead rotation for four hours at 75 rpm, the dispersions were filtered through regenerated cellulose (pore size 0.2 μm), and diluted to 1.5 μg/mL in phosphate buffered saline containing 0.05% (w/v) polysorbate 20. A 96-well microtiter plate, precoated with phosphocholine (CVDefine ELISA kit) was used for the assay.

Non-specific binding was blocked by incubating the plate for 30 minutes with wells filled with a blocking buffer comprising 2% (w/w) bovine serum albumin in the dilution medium. After discarding the blocking buffer, the diluted solutions were added to the wells. The well contents were then then discarded, and the wells washed with dilution medium, according to a standard protocol.

This was followed by addition of solution to the wells containing a secondary antibody (mouse anti-human IgG1) conjugated with horseradish peroxidase. After discarding the well contents, the wells were washed again, followed by addition of a substrate solution containing 3,3′,5,5′-tetramethylbenzidine. After 15 minutes, the reaction was stopped by the addition of sulfuric acid solution (0.5 M), and absorbance in each well was measured using a Tecan Sunrise UV-VIS plate reader equipped with 450 nm optical filters.

### Results

Measuring the absorbance in wells after ELISA showed that the coated ATH3G10, obtained as described in Example 13 retained its affinity compared to uncoated antibodies. The results are shown in FIG. 1 (*p<0.05 as calculated using unpaired two-tailed t-test).

Significance testing using an unpaired two-tailed t-test indicated a small but significant (p=0.02) increase in the affinity after coating (NB. this increase this is likely an artefact stemming from minor uncertainty in the drug load of the coated particles). It was nevertheless concluded however that the process of coating the microparticles as described in Example 13 above does not negatively affect ATH3G10 with regards to affinity to its antigen.

1. A pharmaceutical formulation, comprising a plurality of particles suspended in a carrier system, which particles:
(a) have a weight-, number-, or volume-based mean diameter that is between amount 10 nm and about 700 μm; and
(b) comprise solid cores comprising a biologic active drug, coated, at least in part, by a coating of inorganic material,
and wherein the formulation comprises a concentration of said biologic active drug of at least 50 mg/mL.

2. A pharmaceutical formulation as claimed in claim 1, wherein the concentration of the biologic active drug is at least 250 mg/mL.

3. A pharmaceutical formulation as claimed in claim 1 or claim 2, wherein the concentration of the biologic active drug is at least 450 mg/mL.

4. A pharmaceutical formulation as claimed in any one of the preceding claims, wherein the biologic active drug is selected from an immunoglobulin, a monoclonal antibody, an antibody mimetic, a cytokine, or a cytokine receptor antagonist or agonist.

5. A pharmaceutical formulation as claimed in claim 4, wherein the biologic active drug is an immunoglobulin and is selected from immunoglobulins, normal human, for extravascular administration (J06BA01), immunoglobulins, normal human, for intravascular administration (J06BA02), anti-D (rh) immunoglobulin (J06BB01), tetanus immunoglobulin (J06BB02), human growth hormone, varicella/zoster hepatitis B immunoglobulin (J06BB04), rabies immunoglobulin (J06BB03), immunoglobulin (J06BB05), rubella immunoglobulin (J06BB06), vaccinia immunoglobulin (J06BB07), staphylococcus immunoglobulin (J06BB08), cytomegalovirus immunoglobulin (J06BB09), diphtheria immunoglobulin (J06BB10), hepatitis A immunoglobulin (J06BB11), encephalitis, tick borne immunoglobulin (J06BB12), pertussis immunoglobulin (J06BB13), morbilli immunoglobulin (J06BB14), parotitis immunoglobulin (J06BB15), palivizumab (J06BB16), motavizumab (J06BB17), raxibacumab (J06BB18), bezlotoxumab (J06BB21), obiltoxaximab (J06BB22), anthrax immunoglobulin (J06BB19), combinations (J06BB30), or a mixture of any of these.

6. A pharmaceutical formulation as claimed in claim 4, wherein the biologic active drug is a monoclonal antibody and is selected from edrecolomab (L01XC01), rituximab (L01XC02), trastuzumab (L01XC03), gemtuzumab ozogamicin (L01XC05), cetuximab (L01XC06), bevacizumab (L01XC07), panitumumab (L01XC08), catumaxomab (L01XC09), ofatumumab (L01XC10), ipilimumab (L01XC11), brentuximab vedotin (L01XC12), pertuzumab (L01XC13), trastuzumab emtansine (L01XC14), obinutuzumab (L01XC15), dinutuximab beta (L01XC16), nivolumab (L01XC17), pembrolizumab (L01XC18), blinatumomab (L01XC19), ramucirumab (L01XC21), necitumumab (L01XC22), elotuzumab (L01XC23), daratumumab (L01XC24), mogamulizumab (L01XC25), inotuzumab ozogamicin (L01XC26), olaratumab (L01XC27), durvalumab (L01XC28), bermekimab (L01XC29), avelumab (L01XC31), atezolizumab (L01XC32), cemiplimab (L01XC33), moxetumomab pasudotox (L01XC34), tafasitamab (L01XC35), enfortumab vedotin (L01XC36), polatuzumab vedotin (L01XC37), isatuximab (L01XC38), belantamab mafodotin (L01XC39), dostarlimab (L01XC40), trastuzumab deruxtecan (L01XC41), alemtuzumab (L04AA34), Bi-specific T-cell Engagers (BITE; such as Blinatumomab, Solitomab, AMG 330, MT112, MT111, BAY2010112, MEDI-565, or a mixture of any of these.

7. A pharmaceutical formulation as claimed in claim 4, wherein the biologic active drug is an antibody mimetic and is selected from affibody molecules (such as ABY-025), affilins (such as SPVF 2801), affimers, affitins, alphabodies (such as CMPX-1023), anticalins, avimers, designed ankyrin repeast proteins (DARPins such as MP0112), fynomers, kunitz domain peptides (such as Ecallantide (Kalbitor)), adnectins and monobodies (such as Pegdinetanib (Angiocept)), nanoCLAMPs, single domain antibodies such as camelid antibodies, and V_{NAR} fragments obtained from IgNAR, (immunoglobulin new antigen receptor) from cartilaginous fishes, bivalent single-domain antibodies (such as caplacizumab (Cablivi)); and armadillo repeat proteins hereunder designed armadillo repeat proteins, peptide aptamers, and knottins or a mixture of any of these.

8. A pharmaceutical formulation as claimed in claim 4, wherein the biologic active drug is a human peptide hormone and is selected from amylin, anti-Müllerian hormone, adiponectin, adrenocorticotropic hormone, angiotensinogen, angiotensin, antidiuretic hormone, atrial natriuretic peptide, brain natriuretic peptide, calcitonin, cholecystokinin, corticotropin-releasing hormone, cortistatin, enkephalin, endothelin, erythropoietin, follicle-stimulating hormone, galanin, gastric inhibitory polypeptide, gastrin, ghrelin, glucagon, glucagon-like peptide-1, gonadotropin-releasing hormone, growth hormone-releasing hormone, hepcidin, human chorionic gonadotropin, human placental lactogen, growth hormone, inhibin, insulin, insulin-like growth factor, leptin, lipotropin, luteinizing hormone, melanocyte stimulating hormone, motilin, orexin, osteocalcin, oxytocin, pancreatic polypeptide, parathyroid hormone, Pituitary adenylate cyclase-activating peptide, prolactin, prolactin-releasing hormone, relaxin, renin, secretin, somatostatin, growth hormone-inhibiting hormone, growth hormone release-inhibiting hormone, somatotropin release-inhibiting factor, somatotropin release-inhibiting hormone, thrombopoietin, thyroid-stimulating hormone, thyrotropin, thyrotropin-releasing hormone, vasoactive intestinal peptide, guanylin uroguanylin, tetrakosaktid, mecasermin, somapacitan, pegvisomant, vasopressin, desemopressin, terlipressin, lypressin, ornipressin, argipressin, demoxytocin, carbetocin, gonadorelin, nafarelin, histrelin, ocreotide, anreotide, vapreotide, pasireotide, ganirelix, cetrorelix, elagolix, relugolix, teriparatide, elkatonin

9. A pharmaceutical formulation as claimed in claim 4, wherein the biologic active drug is a cytokine or cytokine antagonist and is selected from IL-1 receptor antagonist, anakinra, IL-2, IL-7. IL-15, IL-21. TNF-alfa, Interferon-gamma.

10. A pharmaceutical formulation as claimed in any one of the preceding claims further comprising a pharmaceutically-acceptable or veterinarily-acceptable adjuvant, diluent, or carrier.

11. A pharmaceutical formulation as claimed in any one of the preceding claims, wherein the carrier system is an aqueous carrier system.

12. A pharmaceutical formulation as claimed in any one of the preceding claims, wherein the coating of inorganic material comprises a mixture of
(i) zinc oxide (ZnO); and
(ii) one or more other metal and/or metalloid oxides,
wherein the atomic ratio ((i):(ii)) is between about 1:6 and up to and including about 6:1,

13. A formulation as claimed in claim 12, wherein the ratio of zinc oxide to other metal and/or metalloid oxides is between about 1:1 and about 6:1.

14. A formulation as claimed in claim 12 or claim 13, wherein the ratio of zinc oxide to other metal and/or metalloid oxides is between about 2:1 and about 5:1.

15. A formulation as claimed in any one of claims 12 to 14, wherein the one or more other metal and/or metalloid oxides are selected from aluminium oxide and/or silicon dioxide.

16. A pharmaceutical formulation as claimed in any one of the preceding claims in the form of a sterile injectable and/or infusible dosage form.

17. A pharmaceutical formulation as claimed in claim 16 in a form that is administrable via a surgical administration apparatus that forms a depot formulation.

18. A process for the preparation of a formulation as defined in any one of the preceding claims, wherein the coated particles are made by applying the layer(s) of mixed oxide coating material to the cores, and/or previously-coated cores, by atomic layer deposition.

19. An injectable and/or infusible dosage form comprising a formulation as defined in any one of claims 1 to 18 contained within a reservoir and an injection or infusion means.

20. A dosage form as claimed in claim 19 which is a surgical administration apparatus that forms a depot formulation.

21. A dosage form as claimed in claim 19 or claim 20, wherein coated particles as defined in any one of claims 1 to 15 and the carrier system are housed separately, and in which admixing occurs prior to and/or during injection or infusion.

22. The use of a formulation as defined in any one of claims 1 to 16 or a dosage form as defined in any one of claims 19 to 21 for the manufacture of a medicament.

23. A formulation for use, a use or a method as claimed in claim 22, wherein, following injection, the formulation provides a depot formulation from which the API is released over a period of time that is between 1 week and about 3 months.

24. A formulation for use, a use or a method as claimed in claim 23 wherein the API is released over a period of time that is between one month and two months.
